(19) United States
(12) Patent Application Publication
(10) Pub. No.: US 2020/0297708 A1
(43) Pub. Date: Sep. 24, 2020

(54) Combination Therapy Using a Chemokine Receptor 2 (CCR2) Antagonist and a PD-1 and/or PD-L1 Inhibitor

(71) Applicants: Jeffrey K. HARRISON, (US); ChemoCentryx, Inc., Mountain View, CA (US)

(72) Inventors: James J. CAMPBELL, San Jose, CA (US); Rajinder SINGH, Belmont, CA (US); Samuel HWANG, Oakland, CA (US); Xueson WU, Oakland, CA (US); Jeffrey K. HARRISON, Gainesville, FL (US)

(21) Appl. No.: 16/824,585

(22) Filed: Mar. 19, 2020

Related U.S. Application Data
(63) Continuation-in-part of application No. 16/358,329, filed on Mar. 19, 2019.
(60) Provisional application No. 62/950,780, filed on Dec. 19, 2019.

(51) Int. Cl. A61K 31/4433; C07K 16/28; A61K 31/496; A61P 35/00; A61K 39/395

(52) U.S. Cl. CPC A61K 31/4433; C07K 16/2827; A61K 39/39533; A61P 35/00; C07K 16/2818; A61K 31/496

(57) ABSTRACT

The present disclosure is drawn to the combination therapy of a Chemokine Receptor 2 (CCR2) antagonist and a PD-1 and/or PD-L1 inhibitor in the treatment of a central nervous system cancer.

This application is a U.S. Non-provisional application claiming priority under 35 U.S.C. 120 and 119(e) to U.S. provisional application No. 62/950,780, filed Dec. 19, 2019, and a continuation-in-part of U.S. application Ser. No. 16/358,329, filed Mar. 19, 2019, the disclosures of which are incorporated herein in their entirety.

### STATEMENT AS TO RIGHTS TO INVENTIONS MADE UNDER FEDERALLY SPONSORED RESEARCH AND DEVELOPMENT

This invention was made with government support under R01NS108781 awarded by the National Institutes of Health and under R35CA197743 awarded by the National Cancer Institute. The government has certain rights in the invention.

### BACKGROUND

Cancerous tumors exploit numerous mechanisms to evade the body's natural cytotoxic immune response such that the tumors are tolerated by the immune system. These mechanisms include dysfunctional T-cell signaling, suppressive regulatory cells, and immune checkpoints that normally act to downregulate the intensity of adaptive immune responses and protect healthy tissues from collateral damage. For instance, tumors develop immune resistance, particularly to T cells that are specific to tumor antigens, by recruiting CCR2⁺ myeloid-derived suppressor cells (MDSCs) and tumor-associated macrophages to the tumors and their surrounding microenvironment.

CCR2⁺ MDSCs have immunosuppressive functions. MDSCs play a key role in a tumor's ability to suppress immune responses. Another key component to this suppression is the activation of immune checkpoints which, in turn, restricts T cell activation and infiltration into tumors. Immune checkpoints refer to inhibitory pathways of the immune system that are essential to maintaining self-tolerance and controlling immune responses in peripheral tissues to minimize collateral tissue damage.

Programmed Death-1 (PD-1) is one of numerous immune checkpoint receptors that are expressed by activated T cells and mediate immunosuppression. Ligands of PD-1 include Programmed Death Ligand-1 (PD-L1) and Programmed Death Ligand-2 (PD-L2) which are expressed on antigen-presenting cells as well as on many human cancer cells. PD-L1 and PD-L2 can downregulate T cell activation and cytokine secretion upon binding to PD-1.

It has been shown that PD-1/PD-L1 interaction inhibitors can mediate potent antitumor activity and are effective for treating some cancers. Despite these findings, there remains a need for an effective treatment for cancers such as solid tumor cancers.

### BRIEF SUMMARY OF THE INVENTION

The present disclosure is drawn to the combination therapy of a Chemokine Receptor 2 (CCR2) antagonist and a PD-1 and/or PD-L1 inhibitor in the treatment of cancer.

In some embodiments, the CCR2 chemokine receptor antagonist has the formula I

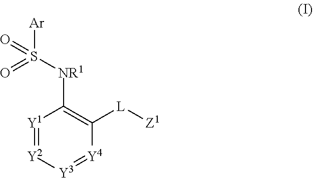

where each variable is described below.

In some embodiments, the CCR2 chemokine antagonist has the formula selected from the group consisting of

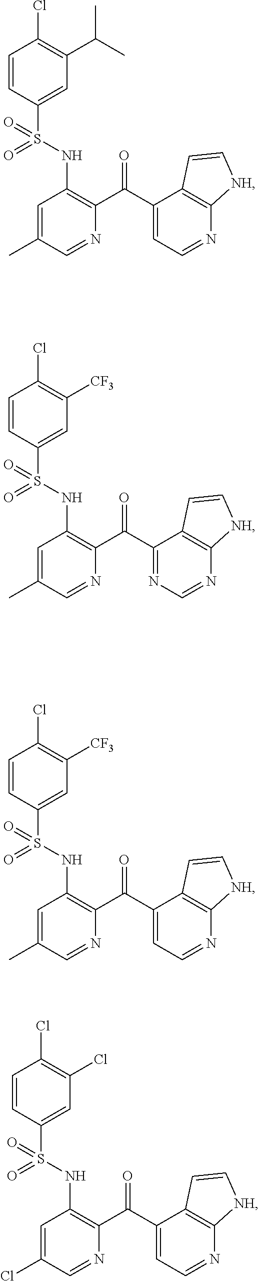 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1 ; O=C(c1ncc(Cl)cc1NS(=O)(=O)c1ccc(Cl)c(Cl)c1)c1ccnc2[nH]ccc12]

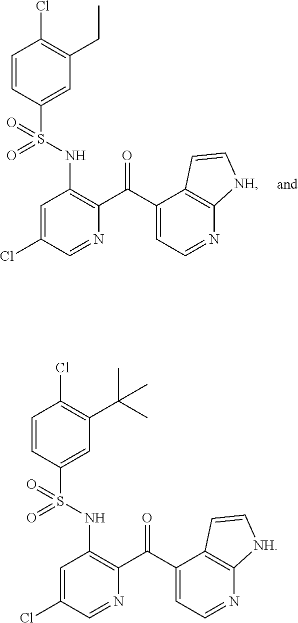 [SMILES: CCc1cc(S(=O)(=O)Nc2cc(Cl)cnc2C(=O)c2ccnc3[nH]ccc23)ccc1Cl ; CC(C)(C)c1cc(S(=O)(=O)Nc2cc(Cl)cnc2C(=O)c2ccnc3[nH]ccc23)ccc1Cl]

or a pharmaceutically acceptable salt thereof.

In some embodiments, the CCR2 antagonist has the formula

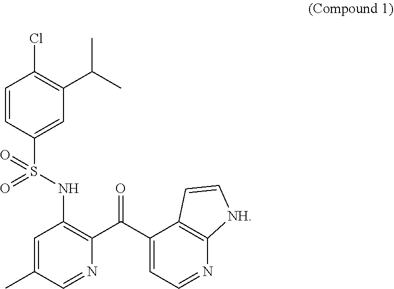 [SMILES: Cc1cnc(C(=O)c2ccnc3[nH]ccc23)c(NS(=O)(=O)c2ccc(Cl)c(C(C)C)c2)c1]

or a pharmaceutically acceptable salt thereof.

In some embodiments, the CCR2 antagonist has the formula

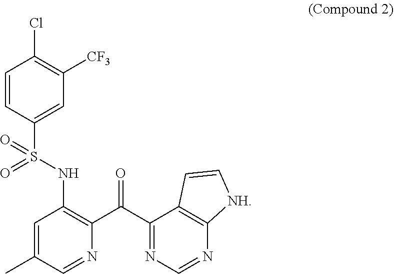 [SMILES: Cc1cnc(C(=O)c2ncnc3[nH]ccc23)c(NS(=O)(=O)c2ccc(Cl)c(C(F)(F)F)c2)c1]

or a pharmaceutically acceptable salt thereof.

In some embodiments, the CCR2 antagonist has the formula

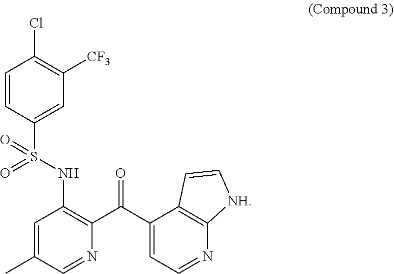 [SMILES: Cc1cnc(C(=O)c2ccnc3[nH]ccc23)c(NS(=O)(=O)c2ccc(Cl)c(C(F)(F)F)c2)c1]

or a pharmaceutically acceptable salt thereof.

In some embodiments, the PD-1 and/or PD-L1 inhibitor is a PD-1 inhibitor.

In some embodiments, the PD-1 inhibitor is selected from the group consisting of pembrolizumab, nivolumab, IBI-308, mDX-400, BGB-108, MEDI-0680, SHR-1210, PF-06801591, PDR-001, GB-226, STI-1110, biosimilars thereof, biobetters thereof, and bioequivalents thereof.

In some embodiments, the PD-1 and/or PD-L1 inhibitor is a PD-L1 inhibitor.

In some embodiments, the PD-L1 inhibitor is selected from the group consisting of durvalumab, atezolizumab, avelumab, BMS-936559, ALN-PDL, TSR-042, KD-033, CA-170, STI-1014, KY-1003, biosimilars thereof, biobetters thereof, and bioequivalents thereof.

In some embodiments, the PD-1 and/or PD-L1 inhibitor is a compound of formula (II)

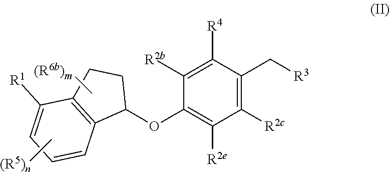

where each variable is described below.

In some embodiments, the cancer is a central nervous system cancer. In some embodiments, the cancer is glioblastoma.

A pharmaceutical combination for treating glioblastoma in a patient is provided. The pharmaceutical combination includes a PD-1 and/or PD-L1 inhibitor; and a compound or a pharmaceutically acceptable salt thereof of formula I:

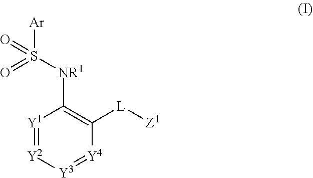

where each variable is described below.

Other objects, features, and advantages of the present invention will be apparent to one of skill in the art from the following detailed description and figures.

### BRIEF DESCRIPTION OF THE DRAWINGS

FIGS. 1A-1E shows distinct cell populations of CCR2 and CX3CR1 expressing myeloid cells in glioma bearing mice. FIG. 1A) Fluorescent images showing representative example of section of KR158 tumor bearing Ccr2^{RFP/WT}/Cx3cr1^{GFP/WT} normal (N) and tumor (T) tissue. Red fluorescence denotes CCR2⁺ cells, while green fluorescence denotes CX3CR1⁺ cells. Image magnification: 20×. FIG. 1B) Flow cytometric analysis of tumor isolates from KR158 (left, n=4) and 005 GSC (right, n=3) tumor bearing Ccr2^{RFP/WT};Cx3cr1^{GFP/WT} mice. Higher CCR2 single positive (p=0.048) and CX3CR1 single positive (p=0.012) cell populations in KR158 versus 005 GSC glioma models are noted. FIG. 1C) Flow cytometric analysis of bone marrow cell populations in CCR2/RFP versus CX3CR1/GFP in naïve (upper left, n=3), mock PBS injected (upper right, n=6), 005 GSC (lower left, n=3), and KR158 (lower right, n=6) tumor bearing Cer2^{RFP/WT};Cx3cr1^{GFP/WT} animals. Quantification shows increase in CCR2 single positive cells in KR158 (p=0.032) and 005 GSC (p=0.001) tumor bearing animals. FIG. 1D) Flow cytometric analysis of tumor isolates from Ccr2^{RFP/WT};Cx3cr1^{GFP/WT} mice. Left panels demonstrate three CD45 populations: negative (left), low (middle), and high (right). Blue arrows denote subpopulations plotted by expression of CCR2 and CX3CR1. CD45^{low} events (upper) a primarily CX3CR1⁺ cell population, while CD45^{hi} events represent a heterogeneous cell population consisting of CCR2⁺, CX3CR1⁺, and CCR2⁻/CX3CR1⁻ cells. FIG. 1E) Flow cytometric analysis of tumor isolates from Ccr2^{RFP/WT};Cx3cr1^{GFP/WT} mice. Left panels represent Ly6C⁺ vs Ly6G⁺ events, and demonstrate three Ly6C populations: negative (bottom), intermediate (middle), and high (top). Blue arrows denote subpopulations plotted by expression of CCR2 and CX3CR1. Ly6C^{hi} events represent a cell population that is primarily CCR2⁺/CX3CR1⁺, while Ly6C⁻ events represent a heterogeneous cell population consisting of CCR2⁺, CX3CR1⁺, and CCR2⁻/CX3CR1⁻ cells. Representative plots shown throughout. *=p<0.05, **=p<0.01

FIGS. 2A-2C show effect of Ccr2 deficiency on glioma bearing mice. FIG. 2A) Survival analysis of KR158 tumor bearing Ccr2^{RFP/WT} and Ccr2^{RFP/RFP} mice treated with or without anti-PD-1. Ccr2 deficiency did not impact survival in control mice (n=8), while anti-PD-1 treatment (n=10) enhanced survival (p=0.035). Triangles mark anti-PD-1 administration. FIG. 2B) Fluorescent imaging of CD11b (green stain) in Ccr2^{RFP/WT} and Ccr2^{RFP/RFP} mice. Representative images shown. FIG. 2C) Fluorescent imaging of femur cross section from Ccr2^{RFP/WT} and Ccr2^{RFP/RFP} naive and KR158 tumor bearing mice. Loss of Ccr2 enhanced CCR2/RFP signal in bone marrow of naive mice (p=0.029), which was further enhanced in tumor bearing Ccr2^{RFP/RFP} animals (p=0.036). Representative images shown. Quantification: average pixel density/cross sectional area from 3 consecutive sections, 3 mice/treatment group. *=p<0.05

FIGS. 3A-3C show impact of Ccr2 deficiency on peripheral and tumor MDSC populations. FIG. 3A) Flow cytometric analysis of RFP⁺ events in Ccr2^{RFP/WT} (n=6) vs. Ccr2^{RFP/RFP} (n=6) mice. Population of RFP⁺ cells within the tumor microenvironment (upper) is reduced (p=0.047), but increased (p=0.024) in bone marrow (lower) of Ccr2 deficient animals. FIG. 3B) Flow cytometric analysis of CD45⁺/CD11b⁺/Ly6C^{hi} events in Ccr2^{RFP/WT} (n=5) vs. Ccr2^{RFP/RFP} (n=5) mice. Population of CD45⁺/CD11b⁺/Ly6C^{hi} cells within the tumor microenvironment (upper) was reduced (p=0.039), but increased (p=0.020) in bone marrow (lower) of Ccr2 deficient animals. FIG. 3C) Quantification of percent RFP⁺ cells that are CD45⁺, CD45⁺/CD11b⁺, and CD45⁺/CD11b⁺/Ly6C^{hi} within bone marrow (left) and tumor (right) in Ccr2^{RFP/WT} (n=5) vs. Ccr2^{RFP/RFP} (n=5) mice. Ratios remain unchanged in bone marrow, but show a significant reduction (p=0.007) of CD45⁺/CD11b⁺/Ly6C^{hi} cells in tumors of Ccr2^{RFP/RFP} vs. Ccr2^{RFP/WT} mice.

FIGS. 4A-4C show effect of combinatorial Compound 3/anti-PD-1 treatment on survival of KR158 and 005 GSC glioma bearing mice. FIG. 4A) Schematic representation of Compound 3 and anti-PD-1 treatment schedules. Survival analysis of FIG. 4B) KR158 (n=8-10) and FIG. 4C) 005 GSC (n=8-10) tumor bearing WT mice treated with Compound 3 and anti-PD-1. In KR158 glioma bearing mice, Compound 3 increased median survival (p=0.002, 32 vs 50 days). Combinatorial treatment increased durable survival (p=0.001). 005 GSC bearing animals had an increase in median survival (p=0.005, 30 vs. 49 days) with combinatorial treatment. Triangles mark anti-PD-1 administration. Brackets indicate Compound 3 administration.

FIG. 5A-5D show impact of combinatorial Compound 3/anti-PD-1 treatment on peripheral and tumor myeloid cell populations. FIG. 5A) Flow cytometric analysis of Ly6C⁺ vs Ly6G⁺ events in KR158 tumor isolates (upper) and bone marrow cell populations (lower) from control (n=6) and Compound 3 (n=6) treated animals. Drug treatment resulted in a reduction (p=0.038) of Ly6C^{hi} events within tumors, and an increase (p=0.028) in bone marrow. FIG. 5B) Flow cytometric analysis of Ly6C⁺ vs Ly6G⁺ events in 005 GSC tumor isolates (upper) and bone marrow cell populations (lower) from control (n=6) and Compound 3 (n=5) treated animals. Drug treatment resulted in a reduction (p=0.015) in Ly6C^{hi} events within tumors, and an increase (p=0.028) in bone marrow. FIG. 5C) Flow cytometric analysis of tumor isolates from KR158 tumor bearing Ccr2^{RFP/WT}/Cx3cr1^{GFP/WT} mice depicting CCR2⁺ vs. CX3CR1⁺ (upper) and Ly6C⁺ vs Ly6G⁺ events (lower) from control (n=5) and Compound 3 (n=7) treated animals. Drug treatment resulted in a significant reduction of CCR2⁺ (p=0.024) and CCR2⁺/CX3CR1⁺ (p=0.032) events. Lower panels report a reduction (p=0.004) in Ly6C^{hi} events within tumors. FIG. 5D) Flow cytometric analysis of tumor isolates from 005 GSC tumor bearing Ccr2^{RFP/WT}/Cx3cr1^{GFP/WT} mice depicting CCR2⁺ vs. CX3CR1⁺ (upper) and Ly6C⁺ vs Ly6G⁺ events (lower) from control (n=6) and Compound 3 (n=6) treated animals. Drug treatment resulted in a reduction of CCR2⁺ (p=0.003), CX3CR1⁺ (p=0.003), and CCR2⁺/CX3CR1⁺ (p=0.0419) events. Lower panels report a reduction (p=0.020) in Ly6C^{hi} events within tumors.

FIGS. 6A-6E show impact of combinatorial Compound 3/anti-PD-1 treatment on CD4⁺ and CD8⁺ T-cells. FIG. 6A) Flow cytometric analysis of CD45⁺/CD3⁺/CD4⁺ and CD8⁺ events within blood extracted from Vehicle/IgG (n=5), Compound 3/IgG (n=3), Vehicle/anti-PD-1 (n=4), or Compound 3/anti-PD-1 (n=3) treated 005 GSC glioma bearing mice. Population of CD45⁺/CD3⁺/CD4⁺ cells remained unchanged in all treatment groups. Representative plots shown throughout. FIG. 6B) Flow cytometric analysis of CD45⁺/CD3⁺/CD4⁺ and CD8⁺ events within draining lymph nodes extracted from Vehicle/IgG (n=6), Compound 3/IgG (n=3), Vehicle/anti-PD-1 (n=5), or Compound 3/anti-PD-1 (n=3) treated 005 GSC glioma bearing mice. Population of CD45⁺/CD3⁺/CD4⁺ cells remained unchanged in all treatment groups. Representative plots shown throughout. FIG. 6C) Flow cytometric analysis of CD45⁺/CD3⁺/CD4⁺ and CD8⁺ events within tumor extracts from Vehicle/IgG (n=7), Compound 3/IgG (n=4), Vehicle/anti-PD-1 (n=6), or Compound 3/anti-PD-1 (n=4) treated 005 GSC glioma bearing mice. The population of CD45⁺/CD3⁺/CD4⁺ cells was significantly increased (p=0.044) with combination Compound 3/anti-PD-1 treatment as compared to control, while the CD45⁺/CD3⁺/CD8⁺ population trended toward increase (p=0.056) between the same groups. Representative plots shown throughout. Flow cytometric analysis of CD45⁺/CD3⁺/PD-1⁺/Tim3⁺/CD4⁺ (FIG. 6D) and CD8⁺ (FIG. 6E) events within tumor extracts from Vehicle/IgG (n=7), Compound 3/IgG (n=4), Vehicle/anti-PD-1 (n=6), or Compound 3/anti-PD-1 (n=4) treated 005 GSC glioma bearing mice. The population of CD45⁺/CD3⁺/PD-1⁺/Tim3⁺/CD4⁺ cells was significantly decreased (p=0.029) with combination Compound 3/anti-PD-1 treatment as compared to control. The CD45⁺/CD3⁺/PD-1⁺/Tim3⁺/CD8⁺ population also decreased (p=0.011) between the same groups.

### DETAILED DESCRIPTION OF THE INVENTION

### Abbreviation and Definitions

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The term “heterocycloalkyl” refers to a cycloalkyl group that contain from one to five heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycyclic ring system. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom. For terms such as cycloalkylalkyl and heterocycloalkylalkyl, it is meant that a cycloalkyl or a heterocycloalkyl group is attached through an alkyl or alkylene linker to the remainder of the molecule. For example, cyclobutylmethyl—is a cyclobutyl ring that is attached to a methylene linker to the remainder of the molecule.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms. Similarly, “alkenylene” and “alkynylene” refer to the unsaturated forms of “alkylene” having double or triple bonds, respectively.

As used herein, a wavy line, “ [symbol] ”, that intersects a single, double or triple bond in any chemical structure depicted herein, represents the point attachment of the single, double, or triple bond to the remainder of the molecule.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the terms “heteroalkenyl” and “heteroalkynyl” by itself or in combination with another term, means, unless otherwise stated, an alkenyl group or alkynyl group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “heteroalkylene” by itself or as part of another substituent means a divalent radical, saturated or unsaturated or polyunsaturated, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group that is attached to the remainder of the molecule (e.g., benzyl, phenethyl, pyridylmethyl and the like).

The above terms (e.g., “alkyl,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below, while the term “alkyl” and related aliphatic radicals is meant to refer to an unsubstituted version, unless indicated to be substituted.

Substituents for the alkyl radicals (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)—NR′, —S(O)R′, —S(O)₂R′, —S(O)₂NR′R″, —NR'S(O)₂R″, —CN and —NO₂ in a number ranging from zero to (2 m′+1), where m′ is the total number of carbon atoms in such radical. R′, R″ and R′″each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to an alkyl radical wherein two substituents on the carbon that is closest to the point of attachment for the radical is replaced with the substituent ═O (e.g., —C(O)CH₃, —C(O)CH₂CH₂OR′ and the like).

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

For the compounds provided herein, a bond that is drawn from a substituent (typically an R group) to the center of an aromatic ring (e.g., benzene, pyridine, and the like) will be understood to refer to a bond providing a connection at any of the available atoms of the aromatic ring. In some embodiments, the depiction will also include connection at a ring which is fused to the aromatic ring. For example, a bond drawn to the center of the benzene portion of an indole, will indicate a bond to any available vertex of the six- or five-membered ring portions of the indole.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. When compounds are provided herein with an identified stereochemistry (indicated as R or S, or with dashed or wedge bond designations), those compounds will be understood by one of skill in the art to be substantially free of other isomers (e.g., at least 80%, 90%, 95%, 98%, 99%, and up to 100% free of the other isomer).

The compounds of the present invention may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, glioblastoma and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “PD-1” or “PD-1 receptor” refers to the programmed death-1 protein, a T-cell co-inhibitor, also known as CD279. The amino acid sequence of the human full-length PD-1 protein is set forth, for example, in GenBank Accession Number NP_005009.2. PD-1 is a 288 amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al., Immunol Rev, 2009, 229(1):356-386). The term “PD-1” includes recombinant PD-1 or a fragment thereof, or variants thereof. The PD-1 receptor has two ligands, PD-ligand-1 (PD-L1) and PD-ligand-2 (PD-L2).

The term “PD-L1” or “programmed death ligand 1” refers to a ligand of the PD-1 receptor also known as CD274 and B7H 1. The amino acid sequence of the human full-length PD-L1 protein is set forth, for example, in GenBank Accession Number NP_054862.1 PD-L1 is a 290 amino acid protein with an extracellular IgV-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, virally-infected cells and autoimmune tissue.

The programmed death 1 (PD-1/PD-L1) pathway acts as a checkpoint to limit T-cell-mediated immune responses. Both PD-1 ligands, PD-L1 and PD-L2, can engage the PD-1 receptor and induce PD-1 signaling and reversible inhibition of T-cell activation and proliferation. When PD-1 ligands on the surface or cancer cells or neighboring cells, these ligands bind to PD-1 receptor positive immune effector cells and utilize the PD-1 pathway to evade an immune response.

The term “immune checkpoint inhibitor” or “immune checkpoint blockade” refers to any agent, molecule, compound, chemical, protein, polypeptide, macromolecule, etc. that blocks or inhibits in a statistically, clinically, or biologically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD-1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+ (αβ) T cells), CD160 (also referred to as BY55) and CGEN-15049. Illustrative immune checkpoint inhibitors include durvalumab (anti-PD-L1 antibody; MEDI4736), pembrolizumab (anti-PD-1 monoclonal antibody), nivolumab (anti-PD-1 antibody), pidilizumab (CT-011; humanized anti-PD-1 monoclonal antibody), AMP224 (recombinant B7-DC-Fc fusion protein), BMS-936559 (anti-PD-L1 antibody), atezolizumab (MPLDL3280A; human Fc-optimized anti-PD-L1 monoclonal antibody), avuelumab (MSB0010718C; human anti-PD-L1 antibody), ipilimumab (anti-CTLA-4 checkpoint inhibitor), tremelimumab (CTLA-4 blocking antibody), and anti-OX40.

The terms “CCR2 antagonist“and” CCR2 chemokine receptor antagonist” are used interchangeably and refer to a small molecule that antagonizes the interaction of the chemokine receptor CCR2 and any one of its ligands. Such a compound could inhibit processes normally triggered by the receptor ligand interaction.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein “mammal” is defined herein to include humans, other primates, cows, sheep, goats, horses, dogs, cats, rabbits, rats, mice and the like. The compounds, agents and compositions described herein are useful for treating a wide variety of cancers including solid tumor cancers.

The term “therapeutically effective amount” means the amount of the subject compound that will elicit the biological or medical response of a cell, tissue, system, or animal, such as a human, that is being sought by the researcher, veterinarian, medical doctor or other treatment provider.

### General

The present disclosure is drawn to the surprising and unexpected finding that combination therapy using a CCR2 antagonist and a PD-1 and/or PD-L1 inhibitor significantly improves cancer treatment as compared to PD-1 and/or PD-L1 inhibition on its own.

Combination Therapy using a CCR2 Antagonist and a PD-1 and/or PD-L1 Inhibitor

Provided herein are methods, compositions, and kits that take advantage of the synergistic effect of CCR2 antagonists and PD-1 and/or PD-L1 inhibitors in treating cancer. A combination treatment that includes both a CCR2 antagonist and PD-1 and/or PD-L1 inhibitor is more effective at treating cancer compared to either compound/antibody alone.

In one aspect, provided herein are methods for treating cancer in a mammal. The method comprises administering to the subject in need thereof a therapeutically effective amount of a CCR2 chemokine receptor antagonist and a therapeutically effective amount of a PD-1 and/or PD-L1 inhibitor.

In some embodiments, the method comprises administering to the subject in need thereof a therapeutically effective amount of a CCR2 chemokine receptor antagonist and a therapeutically effective amount of a PD-1 inhibitor.

In some embodiments, the method comprises administering to the subject in need thereof a therapeutically effective amount of a CCR2 chemokine receptor antagonist and a therapeutically effective amount of a PD-L1 inhibitor.

In some embodiments, the CCR2 chemokine receptor antagonist is a compound of formula I of a subformulae thereof, below. In some embodiments, the CCR2 chemokine receptor antagonist is selected from the group consisting of

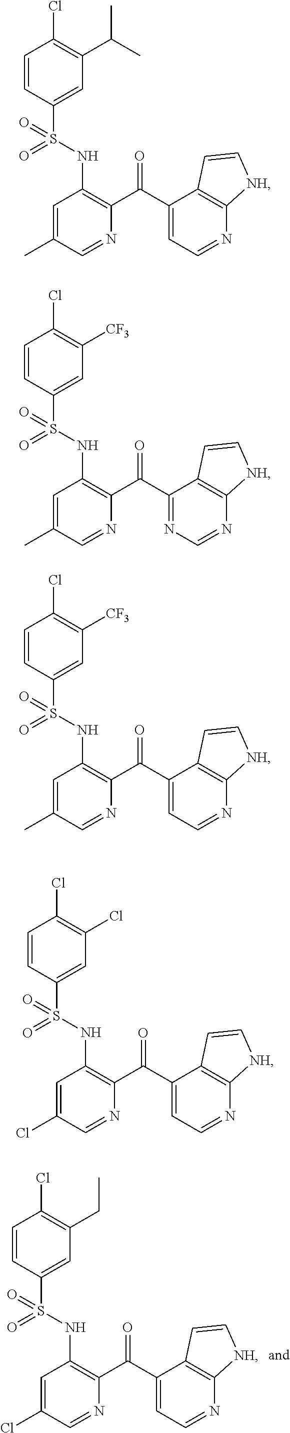

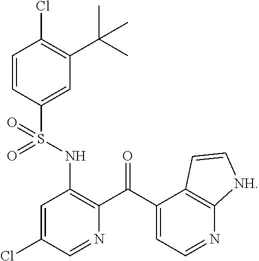 [SMILES: CC(C)(C)c1cc(S(=O)(=O)Nc2cc(Cl)cnc2C(=O)c2ccnc3[nH]ccc23)ccc1Cl]

or a pharmaceutically acceptable salt thereof.

In some embodiments, the PD-1 inhibitor is selected from the group consisting of pembrolizumab, nivolumab, IBI-308, mDX-400, BGB-108, MEDI-0680, SHR-1210, PF-06801591, PDR-001, GB-226, STI-1110, biosimilars thereof, biobetters thereof, and bioequivalents thereof.

In some embodiments, the PD-1 inhibitor is selected from the group consisting of pembrolizumab, nivolumab, IBI-308, mDX-400, BGB-108, MEDI-0680, SHR-1210, PF-06801591, PDR-001, GB-226, and STI-1110.

In some embodiments, the PD-1 inhibitor is RPMI-14.

In some embodiments, the PD-L1 inhibitor is selected from the group consisting of durvalumab, atezolizumab, avelumab, BMS-936559, ALN-PDL, TSR-042, KD-033, CA-170, STI-1014, KY-1003, biosimilars thereof, biobetters thereof, and bioequivalents thereof.

In some embodiments, the PD-L1 inhibitor is selected from the group consisting of durvalumab, atezolizumab, avelumab, BMS-936559, ALN-PDL, TSR-042, KD-033, CA-170, CA-327, STI-1014, KY-1003, biosimilars thereof, biobetters thereof, and bioequivalents thereof.

In some embodiments, the PD-L1 inhibitor is selected from the group consisting of durvalumab, atezolizumab, avelumab, BMS-936559, ALN-PDL, TSR-042, KD-033, CA-327, STI-1014, KY-1003, biosimilars thereof, biobetters thereof, and bioequivalents thereof.

In some embodiments, the PD-L1 inhibitor is selected from the group consisting of durvalumab, atezolizumab, avelumab, BMS-936559, ALN-PDL, TSR-042, KD-033, CA-170, STI-1014, and KY-1003.

In some embodiments, the PD-1 and/or PD-L1 inhibitor is selected from the compounds disclosed in US2015291549, WO16039749, WO15034820, and US2014294898 (BRISTOL MYERS SQUIBB CO) which are thereby incorporated by reference.

In some embodiments, the PD-1 and/or PD-L1 inhibitor is selected from the compounds disclosed in WO14151634, WO15160641, WO16039749, WO16077518, WO16100608, WO16149351, WO2016057624, WO2016100285, US2016194307, US2016222060, and US2014294898 (BRISTOL MYERS SQUIBB CO) which are thereby incorporated by reference.

In some embodiments, the small molecule PD-1 and/or PD-L1 inhibitor is selected from the compounds or pharmaceutical compositions disclosed in WO 2018/005374 filed by ChemoCentryx on Jun. 26, 2017. The contents of which is incorporated herein for all purposes.

In some embodiments, the CCR2 chemokine receptor antagonist and the PD-1 inhibitor or the PD-L1 inhibitor are formulated for concomitant administration.

In other embodiments, the CCR2 chemokine receptor antagonist and the PD-1 inhibitor or the PD-L1 inhibitor are formulated for sequential administration.

In some embodiments, the central nervous system tumor can be a malignant or potentially malignant neoplasm or tissue mass of any size, and includes primary tumors and secondary neoplasms. A solid tumor can be an abnormal growth or mass of tissue that does not contain cysts or liquid areas.

In some embodiments, administering the compounds, agents and compositions of the present invention can decrease or reduce tumor burden, tumor load, tumor size, and/or the number of tumors in a subject. In some cases, the compounds, agents and compositions can prevent or minimize tumor metastasis. In other cases, the compounds, agents and compositions can promote or increase necrosis of the tumor.

In some embodiments, administering the compounds, agents and compositions of the present invention can lead to partial response or complete response (progression-free survival), delay progressive disease, and/or improve overall survival. In some cases, the compounds, agents and compositions can increase the durability of overall response to treatment, promote tumor regression, cancer regression, or disease stabilization, and/or provide a clinical benefit. In other cases, the compounds, agents and compositions can decrease the severity of at least one disease symptom, increase the frequency and duration of disease symptom-free periods, or prevent impairment or disability due to the cancer. In some instances, cancer development or cancer recurrence can be decreased.

Central nervous system cancers include, but are not limited to, neuroblastoma, glioma. astrocytoma, medulloblastoma, craniopharyngioma, ependymoma, pinealoma, hemangioblastoma, acoustic neuroma, oligodendroglioma, meningioma, melanoma, neuroblastoma, and ganglioglioma. In some embodiments, the central nervous system cancer is glioblastoma. The glioma may be characterized as an IDH-mutant type cancer. Examples of astrocytic tumors include, but are not limited to, pilocytic astrocytoma, subependymal giant cell astrocytoma, pleomorphic xanthoastrocytoma, glioblastoma, and anaplastic pleomorphic xanthoastrocytoma. Examples of ependymal tumors include, but are not limited to, subependymoma, myxopapillary ependymoma, ependymoma (RELA fusion-positive), and anaplastic ependymoma. Examples of neuronal and mixed neuronal-glial tumors include, but are not limited to, dysembryoplastic neuroepithelial tumor, gangliocytoma, ganglioglioma, anaplastic ganglioglioma, dysplastic cerebellar gangliocytoma (Lhermitte-Duclos disease), desmoplastic infantile astrocytoma, papillary glioneuronal tumor, rosette-forming glioneuronal tumor, diffuse leptomeningeal glioneuronal tumor, central neurocytoma, extraventricular neurocytoma, cerebellar liponeurocytoma, and paraganglioma.

In some embodiments, the central nervous system cancer may be characterized as being CCR2⁺.

In some embodiments, the administering of the compound of formula I or a pharmaceutically acceptable salt thereof may promote a decrease in CD45^{hi}/CD11b⁺/Ly6C^{hi} cells in a tumor microenvironment and promotes an increase in CD45^{hi}/CD11b⁺/Ly6C^{hi} cells in bone marrow.

In some embodiments, the administering to the patient of the immune checkpoint inhibitor and the compound of formula I or a pharmaceutically acceptable salt thereof may promote an infiltration of a population of T-cells into a tumor microenvironment in the subject. The population of T-cells may comprise a subpopulation of T-cells characterized as being CD45⁺/CD3⁺/CD4⁺. The population of T-cells may comprise a subpopulation of T-cells characterized as being CD45⁺/CD3⁺/CD8⁺.

### CCR2 Antagonists

In some embodiments, the CCR2 antagonist is a small molecule inhibitor of CCR2 having the formula (I):

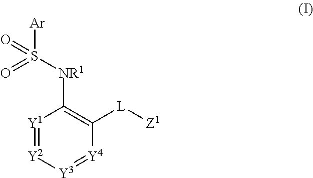

or a pharmaceutically acceptable salt, hydrate, stereoisomer or rotamer thereof; wherein
Ar is selected from the group consisting of substituted or unsubstituted C₆₋₁₀ aryl and substituted or unsubstituted 5- to 10-membered heteroaryl;
R¹ is selected from the group consisting of hydrogen, substituted or unsubstituted C₁₋₈ alkyl, substituted or unsubstituted C₂₋₆ alkenyl, substituted or unsubstituted C₂₋₆ alkynyl, and substituted or unsubstituted 3- to 10-membered heterocyclyl;
Y¹ is selected from the group consisting of —CR^{2a}—, —N—, and —N⁺(O)⁻—;
Y² is selected from the group consisting of —CR^{2b}—, —N—, and —N⁺(O)⁻—;
Y³ is selected from the group consisting of —CR^{2c}—, —N—, and —N⁺(O)⁻—;
R^{2a}, R^{2b}, and R^{2c} are each independently selected from the group consisting of hydrogen, halogen, —CN, —C(O)R³, —CO₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³NR³R⁴, —NR³C(O)R⁴, —NR³C(O)OR⁴, —NR³S(O)₂R⁴, —NR³C(O)NR⁴R⁵, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, and substituted or unsubstituted 5- to 10-membered heteroaryl;
R³, R⁴, and R⁵ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R³ and R⁴, R⁴ and R⁵ or R³ and R⁵ may, together with the atoms to which they are attached, form a substituted or unsubstituted 5-, 6-, or 7-membered ring;
Y⁴ is selected from the group consisting of —N— and —N⁺(O)⁻—;
L is selected from the group consisting of a bond, —O—, —S—, —S(O)—, —S(O)₂—, —CR⁶R⁷, —NR⁸—, —C(O)—, —C(O)NR⁸—, and —NR⁸C(O)—;
R⁶ and R⁷ are each independently selected from the group consisting of hydrogen, halogen, substituted or unsubstituted C₁₋₈ alkyl, substituted or unsubstituted 3- to 10-membered heterocyclyl, substituted or unsubstituted C₂₋₆ alkenyl, substituted or unsubstituted C₂₋₆ alkynyl, —CN, —OR⁹, —NR¹⁰R¹¹, —S(O)R⁹, and —S(O)₂R⁹;
R⁶ and R⁷ may, together with the carbon atom to which they are attached, form substituted or unsubstituted C₃₋₈ cycloalkyl or substituted or unsubstituted 3- to 10-membered heterocyclic ring;
R⁹ is independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R¹⁰ and R¹¹ are each independently selected from the group consisting of substituted or unsubstituted C₁₋₈ alkyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted C₂₋₈ alkenyl, and substituted or unsubstituted C₂₋₈ alkynyl;
R¹⁰ and R¹¹ of —NR¹⁰R¹¹ may, together with the nitrogen, form substituted or unsubstituted 3- to 10-membered heterocyclyl;
R⁸ is selected from the group consisting of hydrogen, C(O)R¹², S(O)₂R¹², CO₂R¹², substituted or unsubstituted C₁₋₈ alkyl, substituted or unsubstituted 3- to 10-membered heterocyclyl, substituted or unsubstituted C₂₋₆ alkenyl, and substituted or unsubstituted C₂₋₆ alkynyl;
R¹² is selected from the group consisting of substituted or unsubstituted C₁₋₈ alkyl, substituted or unsubstituted C₂₋₆ alkenyl, substituted or unsubstituted C₂₋₆ alkynyl, substituted or unsubstituted 3- to 10-membered heterocyclyl, substituted or unsubstituted C₆₋₁₀ aryl, and substituted or unsubstituted 5- to 10-membered heteroaryl;
Z¹ is selected from the group consisting of substituted or unsubstituted C₆₋₁₀ aryl, substituted or unsubstituted 5- to 10-membered heteroaryl, substituted or unsubstituted 3- to 10-membered heterocyclyl, and NR¹³R¹⁴;
R¹³ and R¹⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted (C₁₋₄ alkyl)-(C₆₋₁₀ aryl), and substituted or unsubstituted (C₁₋₄ alkyl)-(5- to 10-membered heteroaryl);
R¹³ and R¹⁴ may, together with the nitrogen, form a substituted or unsubstituted 4-, 5-, 6-, or 7-membered heterocyclyl.

In some embodiments, the CCR2 antagonists are represented by the formula (Ia)

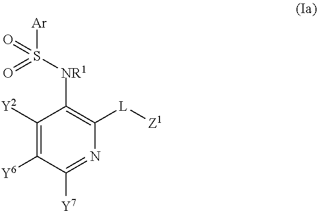

formula (Ia) is a subembodiment of formula (I), wherein
Ar, R¹, L and Z¹ are as defined above
Y⁵, Y⁶ and Y⁷ are each independently selected from the group consisting of hydrogen, halogen, —CN, —C(O)R¹⁵, —CO₂R¹⁵, —C(O)NR¹⁵R¹⁶, —OR¹⁵, —C(O)R¹⁵, —C(O)NR¹⁵R¹⁶, —SR¹⁵, —S(O)R¹⁵, —S(O)₂R¹⁵, —S(O)₂NR¹⁵R¹⁶, —NO₂, —NR¹⁵R¹⁶, —NR¹⁵C(O)R¹⁶, —NR¹⁵C(O)OR¹⁶, —NR¹⁵S(O)₂R¹⁶, —NR¹⁵C(O)NR¹⁶R¹⁷, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, and substituted or unsubstituted 5- to 10-membered heteroaryl;
R¹⁵, R¹⁶ and R¹⁷ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R¹⁵ and R¹⁶, R¹⁶ and R¹⁷ or R¹⁵ and R¹⁷ may, together with the atoms to which they are attached, form a substituted or unsubstituted 5-, 6-, or 7-membered ring.

In some embodiments, the CCR2 antagonists are represented by the formula (Ib)

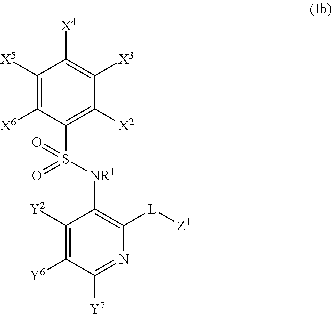

formula (Ib) is a subembodiment of formula (I), wherein
R¹, L and Z¹ are as defined above;
X², X³, X⁴, X⁵, and X⁶ are each independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, —CN, —NO₂, —C(O)R¹⁸, —CO₂R¹⁸, —C(O)NR¹⁸R¹⁹, —R¹⁸, —C(O)R¹⁹, —C(O)NR¹⁸R¹⁹, —NO₂, —NR¹⁸C(O)R¹⁹, —NR¹⁸C(O)NR¹⁹R²⁰, —NR¹⁸R¹⁹, —NR¹⁸CO₂R¹⁹, —NR¹⁸S(O)₂R¹⁹, —SR¹⁸, —S(O)R¹⁸, —S(O)₂R¹⁸, —S(O)₂NR¹⁸R¹⁹, substituted or unsubstituted C₆₋₁₀ aryl, substituted 5- to 10-membered heteroaryl, and substituted or unsubstituted 3- to 10-membered heterocyclyl;
R¹⁸, R¹⁹ and R²⁰ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R¹⁸ and R¹⁹, R¹⁹ and R²⁰ or R¹⁸ and R²⁰ may, together with the atoms to which they are attached, form a substituted or unsubstituted 5-, 6-, or 7-membered ring;
Y⁸, Y⁹ and Y¹⁰ are each independently selected from the group consisting of hydrogen, halogen, —CN, —NO₂, —OR²¹, —CO₂R²¹, —C(O)R²¹, OC(O)NR²¹R²², —C(O)NR²¹R²², —C(O)R²¹, —SR²¹, —S(O)R²¹, —S(O)₂R²¹, —NR²¹R²², —NR²¹C(O)R²², —NR²¹C(O)₂R²², —NR²¹S(O)₂R²², —NR²¹C(O)NR²²R²³, substituted or unsubstituted C₁₋₈ alkyl and substituted or unsubstituted 3- to 10-membered heterocyclyl,
R²¹, R²² and R²³ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R²¹ and R²², R²² and R²³ or R²¹ and R²³ may, together with the atoms to which they are attached, form a substituted or unsubstituted 5-, 6-, or 7-membered ring.

In some embodiments, the CCR2 antagonists are represented by the formula (Ic)

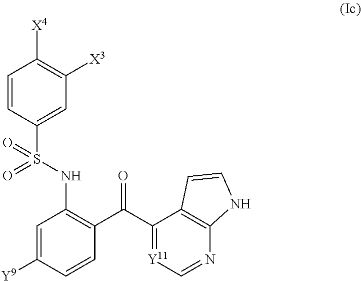

formula (Ic) is a subembodiment of formula (I), wherein
X⁴, X³, and Y⁹ are as defined above; and
Y¹¹ is —CH—, —N—, and N+(O).

In some embodiments, Y¹¹ of formula Ic is —CH—. In some embodiments, Y¹¹ of formula Ic is —N—.

In some embodiments Y⁹ of formula Ib or Ic is selected from the group consisting of hydrogen, halogen, and substituted or unsubstituted C₁₋₈ alkyl.

In some embodiments Y⁹ of Formula Ib or Ic is Cl. In some embodiments Y⁹ of formula Ib or Ic is CH₃.

In some embodiments X⁴ and X³ of formula Ib or Ic are independently selected from the group consisting of hydrogen, halogen, C₁₋₈ alkyl, C₁₋₈ haloalkyl.

In some embodiments, X⁴ of formula Ib or Ic is a halo. In some embodiments, X⁴ of formula Ib or Ic is C₁₋₈ alkyl.

In some embodiments, X⁴ of formula Ib or Ic is a Cl. In some embodiments, X⁴ of formula Ib or Ic is CH₃.

In some embodiments, X³ of formula Ib or Ic is C₁₋₈ haloalkyl. In some embodiments, X³ of formula Ib or Ic is CF₃.

In some embodiments, the CCR2 antagonist has the formula selected from the group consisting of

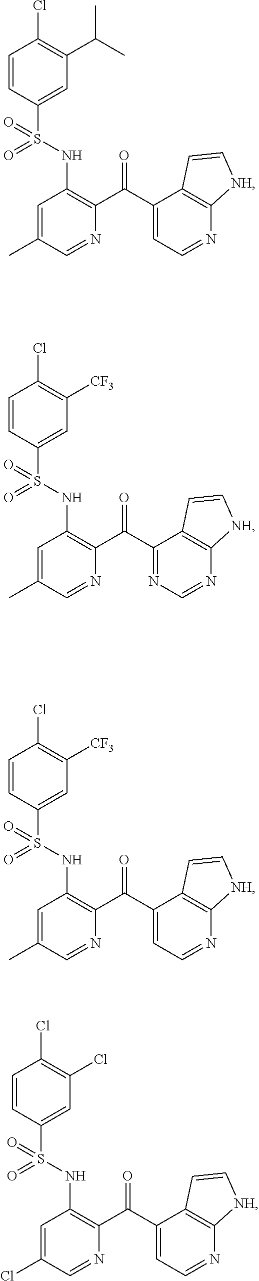 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1 ; O=C(c1ncc(Cl)cc1NS(=O)(=O)c1ccc(Cl)c(Cl)c1)c1ccnc2[nH]ccc12]

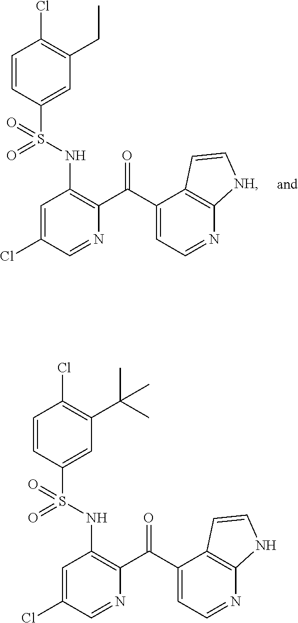 [SMILES: CCc1cc(S(=O)(=O)Nc2cc(Cl)cnc2C(=O)c2ccnc3[nH]ccc23)ccc1Cl ; CC(C)(C)c1cc(S(=O)(=O)Nc2cc(Cl)cnc2C(=O)c2ccnc3[nH]ccc23)ccc1Cl]

or pharmaceutically acceptable salts thereof.

In some embodiments, the CCR2 antagonist has the formula

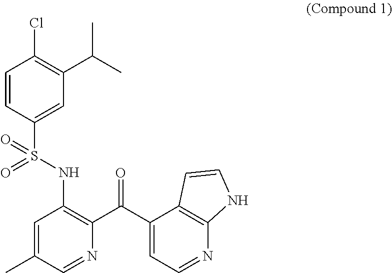 [SMILES: Cc1cnc(C(=O)c2ccnc3[nH]ccc23)c(NS(=O)(=O)c2ccc(Cl)c(C(C)C)c2)c1]

or a pharmaceutically acceptable salt thereof.

In some embodiments, the CCR2 antagonist has the formula

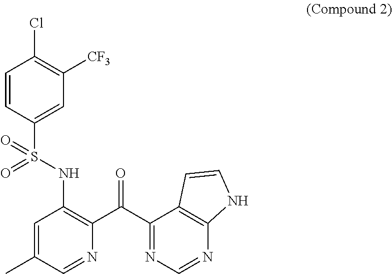 [SMILES: Cc1cnc(C(=O)c2ncnc3[nH]ccc23)c(NS(=O)(=O)c2ccc(Cl)c(C(F)(F)F)c2)c1]

or a pharmaceutically acceptable salt thereof.

In some embodiments, the CCR2 antagonist has the formula

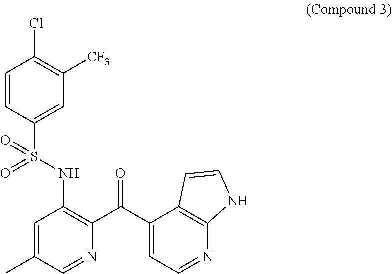 [SMILES: Cc1cnc(C(=O)c2ccnc3[nH]ccc23)c(NS(=O)(=O)c2ccc(Cl)c(C(F)(F)F)c2)c1]

or a pharmaceutically acceptable salt thereof.

### PD-1 Inhibitors and PD-L1 Inhibitors

The methods, compositions, and kits provided herein include immune checkpoint inhibitors such as PD-1/PD-L1 pathway inhibitors (agents). The PD-1 and/or PD-L1 inhibitors of the present invention include small molecules and antibodies.

In some embodiments, a PD-L1 inhibitor can be durvalumab or atezolizumab or avelumab or BMS-936559 (MDX-1105) or ALN-PDL or TSR-042 or KD-033 or CA-170 or CA-327 or STI-1014 or MEDI-0680 or KY-1003.

In some embodiments, a PD-L1 inhibitor can be durvalumab or atezolizumab or avelumab or BMS-936559 (MDX-1105) or ALN-PDL or TSR-042 or KD-033 or CA-170 or STI-1014 or MEDI-0680 or KY-1003. Durvalumab (MEDI4736) is a human monoclonal antibody directed against PD-L1. Atrexolizumab (MPDL3280A) is a fully humanized, engineered IgG1 monoclonal antibody against PD-L1. Avelumab (MSB0010718C) is a fully humanized, engineered IgG1 monoclonal antibody against PD-L1. BMS-936559 (MDX-1105) is a fully human IgG4 monoclonal antibody against PD-L1. ALN-PDL is an inhibitory RNA (RNAi) targeting PD-L1. TSR-042 refers to an engineered chimeric antibody that is directed against the PD-1/PD-L1 pathway. KD-033 refers to a bifunctional anti-PD-L1/IL-15 fusion protein wherein the anti-PD-L1 antibody is linked at its tail to the cytokine IL-15 by the sushi domain of the IL-15 receptor. CA-170 refers to a small molecule antagonist of PD-L1 and VISTA. STI-1014 refers to an anti-PD-L1 antibody. KY-1003 is a monoclonal antibody against PD-L1. CA-327 refers to a small molecule antagonist of PD-L1 and TIM3.

In some embodiments, the PD-1 and/or PD-L1 inhibitor is selected from the group consisting of durvalumab, atezolizumab, pembrolizumab, nivolumab, AP-106, AP-105, MSB-2311, CBT-501, avelumab, AK-105, IO-102, IO-103, PDR-001, CX-072, SHR-1316, JTX-4014, GNS-1480, recombinant humanized anti-PD1 mAb (Shanghai Junshi Biosciences), REGN-2810, pelareorep, SHR-1210, PD1/PDL1 inhibitor vaccine (THERAVECTYS), BGB-A317, recombinant humanized anti-PD-1 mAb (Bio-Thera Solutions), Probody targeting PD-1 (CytomX), XmAb-20717, FS-118, PSI-001, SN-PDL01, SN-PD07, PD-1 modified TILs (Sangamo Therapeutics), PRS-332, FPT-155, jienuo mAb (Genor Biopharma), TSR-042, REGN-1979, REGN-2810, resminostat, FAZ-053, PD-1/CTLA-4 bispecific antibody (MacroGenics), MGA-012, MGD-013, M-7824, PD-1 based bispecific antibody (Beijing Hanmi Pharmaceutical), AK-112, AK-106, AK-104, AK-103, BI-754091, ENUM-244C₈, MCLA-145, MCLA-134, anti-PD1 oncolytic monoclonal antibody (Transgene SA), AGEN-2034, IBI-308, WBP-3155, JNJ-63723283, MEDI-0680, SSI-361, CBT-502, anti-PD-1 bispecific antibody, dual targeting anti-PD-1/LAG-3 mAbs (TESARO), dual targeting anti-PD-1/TIM-3 mAbs (TESARO), PF-06801591, LY-3300054, BCD-100, STI-1110, pembrolizumab biosimilar, nivolumab biosimilar, PD-L1-TGF-beta therapy, KY-1003, STI-1014, GLS-010, AM-0001, GX-P2, KD-033, PD-L1/BCMA bispecific antibody (Immune Pharmaceuticals), PD-1/Ox40 targeting bispecific antibody (Immune Pharmaceuticals), BMS-936559, anti-PD-1/VEGF-A DARPins (Molecular Partners), mDX-400, ALN-PDL, PD-1 inhibitor peptide (Aurigene), siRNA loaded dendritic cell vaccine (Alnylam Pharmaceuticals), GB-226, PD-L1 targeting CAR-TNK-based immunotherapy (TNK Therapeutics/NantKwest), INSIX RA, INDUS-903, AMP-224, anti-CTLA-4/anti-PD-1 bispecific humanized antibody (Akeso Biopharma), B7-H1 vaccine (State Key Laboratory of Cancer Biology/Fourth Military Medical University), and GX-D1.

In some embodiments, a PD-1 inhibitor can be pembrolizumab or nivolumab or IBI-308 or mDX-400 or BGB-108 or MEDI-0680 or SHR-1210 or PF-06801591 or PDR-001 or GB-226 or STI-1110. Nivolumab (also known as OPDIVO™, MDX-1106, BMS-936558, and ONO-4538) is a human IgG4 monoclonal antibody against PD-1. Pembrolizumab (also known as KEYTRUDA®, lambrolizumab, and MK-34) is a humanized IgG4 kappa isotype monoclonal antibody against PD-1. IBI-308 refers to a monoclonal antibody directed to PD-1. mDX-400 refers to a mouse antibody against PD-1. BGB-108 is a humanized monoclonal antibody against PD-1. MEDI-0680 (AMP-514) is a humanized IgG4 monoclonal antibody against PD-1. SHR-1210 refers to a monoclonal antibody against PD-1. PF-06801591 is a monoclonal antibody against PD-1. PDR-001 refers to a monoclonal antibody against PD-1. GB-226 refers to a monoclonal antibody against PD-1. STI-1110 refers to a monoclonal antibody against PD-1.

In some embodiments, the PD-1 inhibitor is RPMI-14.

In some embodiments, the PD-1 inhibitor is an antibody selected from Nivolumab, Pembrolizumab, and Pidilizumab.

The anti-PD-1 antibodies, and antibody fragments described herein encompass proteins having amino acid sequences that vary from those of the described antibodies, but that retain the ability to bind PD-1.

In some embodiments, the anti-PD-1 antibodies include bispecific antibodies and antibody-like therapeutic proteins including DARTs®, DUOBODIES®, BITES®, XmAbs®, TandAbs®, Fab derivatives, and the like that bind to PD-1.

The anti-PD-L1 antibodies and antibody fragments described herein encompass proteins having amino acid sequences that vary from those of the described antibodies, but that retain the ability to bind PD-L1. Such variant antibodies and fragments thereof can comprise one or more additions, deletions, or substitutions of amino acids when compared to the parent sequence, but exhibit biological activity that is essentially equivalent or essentially bioequivalent to that of the described antibodies.

In some embodiments, the anti-PD-L1 antibodies include bispecific antibodies and antibody-like therapeutic proteins including DARTs®, DUOBODIES®, BITES®, XmAbs®, TandAbs®, Fab derivatives, and the like that bind to PD-L1.

Non-limiting examples of additional PD-1/PD-L1 pathway inhibitors are described in, e.g., Chen and Han, Jour Clin Invest, 2015, 125(9):3384-3391, U.S. Pat. Nos. 8,168,757; 8,354,509; 8,552,154; 8,741,295; and 9,212,224; U.S. Patent App. Publ. Nos. 2014/0341917; 2015/0203580 and 2015/0320859; International Patent App. Publ. No. WO2015/026634.

A biological product, e.g., an antibody or a fragment thereof, is considered a biosimilar if, for example, the biological product is highly similar to an already FDA-approved biological product, known as the reference product. A biosimilar has no clinically meaningful differences in terms of safety and effectiveness from the reference product. A biosimilar can also have the same mechanism of action, route of administration, dosage form, and strength as its reference product.

Two biological products, e.g., antibodies or fragments thereof, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In some embodiments, two biological products (e.g., two antibodies or fragments thereof) are bioequivalent if there are no clinically meaningful differences in their safety, purity, or potency.

In other embodiments, two biological products (e.g., two antibodies or fragments thereof) are bioequivalent if a patient can be switched one or more times between the reference product and the biological product without an expected increase in the risk of adverse effects, including a clinically significant change in immunogenicity, or diminished effectiveness, as compared to continued therapy without such switching.

In yet other embodiments, two biological products (e.g., two antibodies or fragments thereof) are bioequivalent if they both act by a common mechanism of action for the condition of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Biobetter variants of the antibodies described herein may be based on an existing reference antibody specific for an target antigen, e.g., PD-1 or PD-L1, which has undergone changes such that, for example, it has a higher binding affinity to its target antigen and/or binds to a different epitope than the reference antibody, or has more desirable therapeutic efficacy, expression and/or biophysical characteristics.

In some embodiments, the PD-1 and/or PD-L1 inhibitor is a small molecule PD-1 and/or PD-L1 inhibitor, or a pharmaceutically acceptable salt thereof, of the formula:

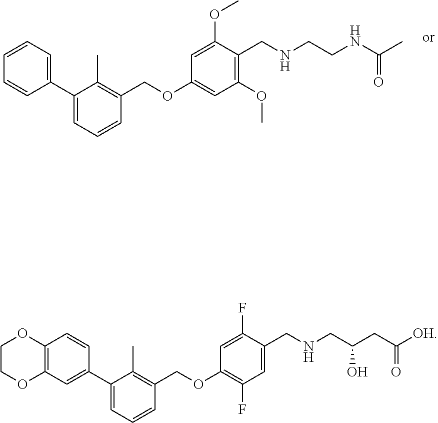 [SMILES: COc1cc(OCc2cccc(-c3ccccc3)c2C)cc(OC)c1CNCCNC(C)=O ; Cc1c(COc2cc(F)c(CNC[C@@H](O)CC(=O)O)cc2F)cccc1-c1ccc2c(c1)OCCO2]

In some embodiments, the PD-1 and/or PD-L1 inhibitor is a small molecule PD-1/PD-L1 inhibitor having the formula (II)

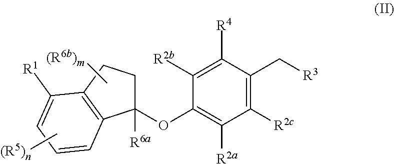

or a pharmaceutically acceptable salt thereof; wherein:
R¹ is selected from the group consisting of halogen, C₅₋₈ cycloalkyl, C₆₋₁₀ aryl and thienyl, wherein the C₆₋₁₀ aryl and thienyl are optionally substituted with 1 to 5 R^{x} substituents;
each R″ is independently selected from the group consisting of halogen, —CN, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}—C(O)NR^{a}R^{b}, —NR^{a}R^{b}, —OR^{a}, —O—X—O, —O—X¹—CO₂R^{a}, —O—X¹—CONR^{a}R^{b}, —X¹—OR^{a}, —X¹—NR^{a}R^{b}, —X¹—CO₂R^{a}, —X¹—CONR^{a}R^{b}, —SF₅, and —S(O)₂NR^{a}R^{b}, wherein each X¹ is a C₁₋₄ alkylene; each R^{a} and R^{b} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, wherein the five or six-membered ring is optionally substituted with oxo; each R^{c} is independently selected from the group consisting of C₁₋₈ alkyl, C₂₋₈ alkenyl, C₂₋₈ alkynyl and C₁₋₈ haloalkyl; and optionally when two R^{x} substituents are on adjacent atoms, they are combined to form a fused five, six or seven-membered carbocyclic or heterocyclic ring optionally substituted with from 1 to 3 substituents independently selected from halo, oxo, C₁₋₈ haloalkyl and C₁₋₈ alkyl;
each R^{2a}, R^{2b} and R^{2c} is independently selected from the group consisting of H, halogen, —CN, —R^{d}, —CO₂R^{e}, —CONR^{e}R^{f}, —C(O)R^{e}, —OC(O)NR^{e}R^{f}, —NR^{f}C(O)R^{e}, —NR^{f}C(O)₂R^{d}, —NR^{e}—C(O)NR^{e}R^{f}, —NR^{e}R^{f}, —OR^{e}, —O—X²—OR^{e}, —O—X²—NR^{e}R^{f}, —O—X²—CO₂R^{e}, —O—X²—CONR^{e}R^{f}, —X²—OR^{e}, —X²—NR^{e}R^{f}, —X²—CO₂R^{e}, —X²—CONR^{e}R^{f}, —SF₅, —S(O)₂NR^{e}R^{f}, C₆₋₁₀ aryl and C₅₋₁₀ heteroaryl, wherein each X² is a C₁₋₄ alkylene; each R^{e} and R^{f} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O and S, and optionally substituted with oxo; each R^{d} is independently selected from the group consisting of C₁₋₈ alkyl, C₂₋₈ alkenyl, and C₁₋₈ haloalkyl;
R³ is selected from the group consisting of —NR^{g}R^{h} and C₄₋₁₂ heterocyclyl, wherein the C₄₋₁₂ heterocyclyl is optionally substituted with 1 to 6 R^{y};
each R^{y} is independently selected from the group consisting of halogen, —CN, —Rⁱ, —CO₂R^{j}, —CONR^{j}R^{k}, —CONHC₁₋₆ alkyl-OH, —C(O)R, —OC(O)NR^{j}R^{k}, —NR^{j}C(O)R^{k}, —NR^{j}C(O)₂R^{k}, CONOH, PO₃H₂, —NR¹—C₁₋₆ alkyl-C(O)₂R^{k}, —NR^{l}C(O)NR^{j}R^{k}, —NR^{j}R^{k}, —OR, —S(O)₂NR^{j}R^{k}, —O—C₁₋₆alkyl-OR^{j}, —O—C₁₋₆ alkyl-NR^{j}R^{k}, —O—C₁₋₆ alkyl-CO₂R^{j}, —O—C₁₋₆ alkyl-CONR^{j}R^{k}, —C₁₋₆ alkyl-OR, —C₁₋₆ alkyl-NR^{j}R^{k}, —C₁₋₆ alkyl-CO₂R^{j}, —C₁₋₆ alkyl-CONR^{j}R^{k}, and SF₅,
wherein the C₁₋₆ alkyl portion of R^{y} is optionally further substituted with OH, SO₂NH₂, CONH₂, CONOH, PO₃H₂, COO—C₁₋₈alkyl or CO₂H, wherein each R^{j} and R^{k} is independently selected from hydrogen, C₁₋₈ alkyl optionally substituted with 1 to 2 substituents selected from OH, SO₂NH₂, CONH₂, CONOH, PO₃H₂, COO—C₁₋₈alkyl or CO₂H, and C₁₋₈ haloalkyl optionally substituted with 1 to 2 substituents selected from OH, SO₂NH₂, CONH₂, CONOH, PO₃H₂, COO—C₁₋₈alkyl or CO₂H, or when attached to the same nitrogen atom R^{j} and R^{k} can be combined with the nitrogen atom to form a five or six-membered ring having from 0 to 2 additional heteroatoms as ring members selected from N, O or S, and optionally substituted with oxo; each Rⁱ is independently selected from the group consisting of —OH, C₁₋₈ alkyl, C₂₋₈ alkenyl, and C₁₋₈ haloalkyl each of which may be optionally substituted with OH, SO₂NH₂, CONH₂, CONOH, PO₃H₂, COO—C₁₋₈alkyl or CO₂H;
R^{g} is selected from the group consisting of H, C₁₋₈ haloalkyl and C₁₋₈ alkyl;
R^{h} is selected from —C₁₋₈ alkyl, C₁₋₈ haloalkyl, C₁₋₈ alkyl-COOH, C₁₋₈ alkyl-OH, C₁₋₈ alkyl-CONH₂, C₁₋₈ alkyl-SO₂NH₂, C₁₋₈ alkyl-PO₃H₂, C₁₋₈ alkyl-CONOH, C₁₋₈ alkyl-NR^{h1}R^{h2}, —C(O)—C₁₋₈ alkyl, —C(O)—C₁₋₈alkyl-OH, —C(O)—C₁₋₈alkyl-COOH, C₃₋₁₀ cycloalkyl, —C₃₋₁₀ cycloalkyl-COOH, —C₃₋₁₀ cycloalkyl-OH, C₄₋₈ heterocyclyl, —C₄₋₈ heterocyclyl-COOH, —C₄₋₈ heterocyclyl-OH, —C₁₋₈ alkyl-C₄₋₈ heterocyclyl, —C₁₋₈ alkyl-C₃₋₁₀ cycloalkyl, C₅₋₁₀ heteroaryl, —C₁₋₈ alkyl-C₅₋₁₀ heteroaryl, C₁₀ carbocyclyl, —C₁₋₈ alkyl-C₆₋₁₀ aryl, —C₁₋₈ alkyl-(C═O)—C₆₋₁₀ aryl, —C₁₋₈ alkyl-NH(C═O)—C-s alkenyl, —C₁₋₈ alkyl-NH(C═O)—C₁₋₈ alkyl, —C₁₋₈ alkyl-NH(C═O)—C₁₋₈ alkynyl, —C₁₋₈ alkyl-(C═O)—NH—C₁₋₈ alkyl-COOH, and —C₁₋₈ alkyl-(C═O)—NH—C₁₋₈ alkyl-OH optionally substituted with CO₂H; or
   R^{h} combined with the N to which it is attached is a mono-, di- or tri-peptide comprising 1-3 natural amino acids and 0-2 non-natural amino acids, wherein
   the non-natural aminoacids have an alpha carbon substituent selected from the group consisting of C₂₋₄ hydroxyalkyl, C₁₋₃ alkyl-guanidinyl, and C₁₋₄ alkyl-heteroaryl,
   the alpha carbon of each natural or non-natural amino acids are optionally further substituted with a methyl group, and
   the terminal moiety of the mono-, di-, or tri-peptide is selected from the group consisting of C(O)OH, C(O)O—C₁₋₆ alkyl, and PO₃H₂, wherein
   R^{h1} and R^{h2} are each independently selected from the group consisting of H, C₁₋₆ alkyl, and C₁₋₄ hydroxyalkyl;
   the C₁₋₈ alkyl portions of R^{h} are optionally further substituted with from 1 to 3 substituents independently selected from OH, COOH, SO₂NH₂, CONH₂, CONOH, COO—C₁₋₈ alkyl, PO₃H₂ and C₅-6 heteroaryl optionally substituted with 1 to 2 C₁₋₃ alkyl substituents,
   the C₁₀ carbocyclyl, C₅₋₁₀ heteroaryl and the C₆₋₁₀ aryl portions of R^{h} are optionally substituted with 1 to 3 substituents independently selected from OH, B(OH)₂, COOH, SO₂NH₂, CONH₂, CONOH, PO₃H₂, COO—C₁₋₈ alkyl, C₁₋₄ alkyl, C₁₋₄ alkyl-OH, C₁₋₄ alkyl-SO₂NH₂, C₁₋₄alkyl CONH₂, C₁₋₄ alkyl-CONOH, C₁₋₄ alkyl-PO₃H₂, C₁₋₄ alkyl-COOH, and phenyl and
   the C₄₋₈ heterocyclyl and C₃₋₁₀ cycloalkyl portions of R^{h} are optionally substituted with 1 to 4 R^{w} substituents;
each R^{w} substituent is independently selected from C₁₋₄ alkyl, C₁₋₄ alkyl-OH, C₁₋₄ alkyl-COOH, C₁₋₄ alkyl-SO₂NH₂, C₁₋₄ alkyl CONH₂, C₁₋₄ alkyl-CONOH, C₁₋₄ alkyl-PO₃H, OH, COO—C₁₋₈ alkyl, COOH, SO₂NH₂, CONH₂, CONOH, PO₃H₂ and oxo;
R⁴ is selected from the group consisting of O—C₁₋₈ alkyl, O—C₁₋₈ haloalkyl, O—C₁₋₈ alkyl-R^{z}, C₆₋₁₀ aryl, C₅₋₁₀ heteroaryl, —O—C₁₋₄ alkyl-C₆₋₁₀ aryl and —O—C₁₋₄ alkyl-C₅₋₁₀ heteroaryl, wherein the C₆₋₁₀ aryl and the C₅₋₁₀ heteroaryl are optionally substituted with 1 to 5 R^{z};
each R^{z} is independently selected from the group consisting of halogen, —CN, —R^{m}, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X³—OR, —O—X³—NRⁿR^{p}, —O—X³—CO₂Rⁿ, —O—X³—CONRⁿR^{p}, —X³—ORⁿ, —X³—NRⁿR^{p}, —X³—CO₂Rⁿ, —X³—CONRⁿR^{p}, —SF₅, —S(O)₂RⁿR^{p}, —S(O)₂NRⁿR^{p}, and three to seven-membered carbocyclic or four to seven-membered heterocyclic ring wherein the three to seven-membered carbocyclic or four to seven-membered heterocyclic ring is optionally substituted with 1 to 5 R^{t}, wherein each R^{t} is independently selected from the group consisting of C₁₋₈ alkyl, C₁₋₈haloalkyl, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X³—ORⁿ, —O—X³—NRⁿR^{p}, —O—X³—CO₂Rⁿ, —O—X³—CONRⁿR^{p}, —X³—ORⁿ, —X³—NRⁿR^{p}, —X³—CO₂Rⁿ, —X³—CONRⁿR^{p}, —SF₅, and —S(O)₂NRⁿR^{p};
wherein each X³ is a C₁₋₄ alkylene; each Rⁿ and R^{p} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R^{m} is independently selected from the group consisting of C₁₋₈ alkyl, C₂₋₈ alkenyl, and C₁₋₈ haloalkyl; and optionally when two R^{z} substituents are on adjacent atoms, they are combined to form a fused five or six-membered carbocyclic or heterocyclic ring optionally substituted with oxo;
each n is independently 0, 1, 2 or 3;
each R⁵ is independently selected from the group consisting of halogen, —CN, —R^{q}, —CO₂R^{r}, —CONR^{r}R^{s}, —C(O)R^{r}, —OC(O)NR^{r}R^{s}, —NR^{r}C(O)R^{s}, —NR^{r}C(O)₂R^{q}, —NR^{r}—C(O)NR^{r}R^{s}, —NR^{r}R^{s}, —OR^{r}, —O—X⁴—OR^{r}, —O—X⁴—NR^{r}R^{s}, —O—X⁴—CO₂R^{r}, —O—X⁴—CONR^{r}R^{s}, —X⁴—OR^{r}, —X⁴—NR^{r}R^{s}, —X⁴—CO₂R^{r}, —X⁴—CONR^{r}R^{s}, —SF₅, —S(O)₂NR^{r}R^{s}, wherein each X⁴ is independently a C₁₋₄ alkylene; each R^{r} and R^{s} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R″ is independently selected from the group consisting of C₁₋₈ alkyl, and C₁₋₈ haloalkyl;
R^{6a} is selected from the group consisting of H, C₁₋₄ alkyl and C₁₋₄ haloalkyl;
each R^{6b} is independently selected from the group consisting of F, C₁₋₄ alkyl, O—R^{u}, C₁₋₄ haloalkyl, NR^{u}R^{v}, wherein each R^{u} and R^{v} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; and
m is 0, 1, 2, 3 or 4.

In some embodiments, the small molecule PD-1/PD-L1 inhibitor is selected from the compounds or pharmaceutical compositions disclosed in WO 2018/005374 filed by ChemoCentryx on Jun. 26, 2017. The contents of which is incorporated herein for all purposes.

The PD-1 and/or PD-L1 inhibitors of the present disclosure can be formulated to retard the degradation of the compound or antibody or to minimize the immunogenicity of the antibody. A variety of techniques are known in the art to achieve these purposes.

### Pharmaceutical Compositions

The pharmaceutical compositions provided herein, such as those including compounds for modulating CCR2 activity and agents for blocking the PD-1/PD-L1 pathway can contain a pharmaceutical carrier or diluent.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

Biological products such as antibodies of the present invention may be constituted in a pharmaceutical composition containing one or antibodies or a fragment thereof and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and nonaqueous carriers, and/or adjuvants such as preservatives, wetting agents, emulsifying agents and dispersing agents.

The pharmaceutical compositions for the administration of the compounds and agents of this invention may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Pat. No. 6,451,339, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example, PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and agents of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present invention are employed. As used herein, topical application is also meant to include the use of mouth washes and gargles.

The compounds of this invention may also be coupled a carrier that is a suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the invention may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the invention, the compound of the invention is coupled to a polymer or semipermeable polymer matrix that is formed as a stent or stent-graft device.

The compounds and agents of the invention may be formulated for depositing into a medical device, which may include any of variety of conventional grafts, stents, including stent grafts, catheters, balloons, baskets or other device that can be deployed or permanently implanted within a body lumen. As a particular example, it would be desirable to have devices and methods which can deliver compounds of the invention to the region of a body which has been treated by interventional technique. For instance, the compound and agent can be delivers to the tumor or the microenvironment surrounding the tumor.

The term “deposited” means that the compound and agent are coated, adsorbed, placed, or otherwise incorporated into the device by methods known in the art. For example, the compound and agent may be embedded and released from within (“matrix type”) or surrounded by and released through (“reservoir type”) polymer materials that coat or span the medical device. In the later example, the compound and agent may be entrapped within the polymer materials or coupled to the polymer materials using one or more the techniques for generating such materials known in the art. In other formulations, the compound and agent may be linked to the surface of the medical device without the need for a coating by means of detachable bonds and release with time, can be removed by active mechanical or chemical processes, or are in a permanently immobilized form that presents the inhibitory agent at the implantation site.

In one embodiment, the compound and agent may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but a bioabsorbable polymer is preferred for this embodiment since, unlike a biostable polymer, it will not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D,L-lactic acid), poly(D,L-lactide) (PLA), poly(L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable popolymers known in the art. Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

In some embodiments, the compound and agent are formulated for release from the polymer coating into the environment in which the medical device is placed. Preferably, the compound and agent are released in a controlled manner over an extended time frame (e.g., weeks or months) using at least one of several well-known techniques involving polymer carriers or layers to control elution. Some of these techniques were previously described in U.S. Patent App. Publ. No. 20040243225.

### Methods of Administration of Combination Therapy

In another aspect, the present disclosure provides a combination therapy for the treatment of cancer. The combination therapy includes a therapeutically effective amount of a CCR2 antagonist and a therapeutically effective amount of a PD-1 and/or PD-L1 inhibitor. The combination of therapeutic agents can act synergistically to effect the treatment or prevention of cancer.

Depending on the disease status and the subject's condition, the compounds, antibodies, and formulations of the present disclosure may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration. In addition, the compounds and antibodies may be formulated, alone or together, in suitable dosage unit formulations containing conventional nontoxic pharmaceutically acceptable carriers, adjuvants and vehicles appropriate for each rouse of administration. The present disclosure also contemplates administration of the compounds and antibodies of the present disclosure in a depot formulation.

It will be understood, that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In the treatment of cancers, e.g., solid tumors which require chemokine receptor modulation, an appropriate dosage level of a CCR2 antagonist will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

An appropriate dosage level of a PD-1 inhibitor and/or a PD-L1 inhibitor will generally be about 0.0001 to about 100 mg/kg, usually from about 0.001 to about 20 mg/kg, and more usually from about 0.01 to about 10 mg/kg, of the subject's body weight. Preferably, the dosage is within the range of 0.1-10 mg/kg body weight. For example, dosages can be 0.1, 0.3, 1, 3, 5 or 10 mg/kg body weight, and more preferably, 0.3, 1, 3, or 10 mg/kg body weight. The dosing schedule can typically be designed to achieve exposures that result in sustained receptor occupancy (RO) based on typical pharmacokinetic properties of an antibody. An exemplary treatment regime of anitibodies entails administration once per week, once every two weeks, once every three weeks, once every four weeks, once a month, once every 3 months or once every three to 6 months. For example, a dosing schedule may comprise administering an antibody: (i) every two weeks in 6-week cycles; (ii) every four weeks for six dosages, then every three months; (iii) every three weeks; (iv) 3-10 mg/kg body weight once followed by 1 mg/kg body weight every 2-3 weeks. Considering that an IgG4 antibody typically has a half-life of 2-3 weeks, a preferred dosage regimen for an anti-PD-1 or anti-PD-L1 antibody comprises 0.3-10 mg/kg body weight, preferably 3-10 mg/kg body weight, more preferably 3 mg/kg body weight via intravenous administration, with the antibody being given every 14 days in up to 6-week or 12-week cycles until complete response or confirmed progressive disease. An exemplary treatment regime of small molecules entails administration daily, twice per week, three times per week, or once per week. The dosage and scheduling may change during a course of treatment.

In some embodiments, two or more antibodies with different binding specificities are administered simultaneously, in which case the dosage of each antibody administered falls within the ranges indicated. The antibody can be administered on multiple occasions. Intervals between single dosages can be, for example, weekly, every 2 weeks, every 3 weeks, monthly, every three months or yearly. Intervals can also be irregular as indicated by measuring blood levels of antibody to the target antigen in the patient. In some methods, dosage is adjusted to achieve a plasma antibody concentration of about 1-1000 mg/ml and in some methods about 25-300 mg/ml.

The therapeutic compound and agent in the combination therapy disclosed herein may be administered either alone or in a pharmaceutical composition which comprises the therapeutic compound and agent and one or more pharmaceutically acceptable carriers, excipients and diluents.

In some embodiments, the therapeutic compound and agent are each provided in an amount that would be sub-therapeutic if provided alone or without the other. Those of skill in the art will appreciate that “combinations” can involve combinations in treatments (i.e., two or more drugs can be administered as a mixture, or at least concurrently or at least introduced into a subject at different times but such that both are in a subject at the same time).

Likewise, compounds, agents and compositions of the present invention may be used in combination with other drugs that are used in the treatment, prevention, suppression or amelioration of cancer. Such other drugs may be administered, by a route and in an amount commonly used therefor, contemporaneously or sequentially with a compound, agent or composition of the present invention. When a compound, agent or composition of the present invention is used contemporaneously with one or more other drugs, a pharmaceutical composition containing such other drugs in addition to the compound, agent or composition of the present invention is preferred. Accordingly, pharmaceutical compositions can include those that also contain one or more other active ingredients or therapeutic agents, in addition to a compound, agent or composition of the present invention.

Combination therapy includes co-administration of the CCR2 antagonist and the PD-1 and/or PD-L1 inhibitor, sequential administration of the CCR2 antagonist and the PD-1 and/or PD-L1 inhibitor, administration of a composition containing the CCR2 antagonist and the PD-1 and/or PD-L1 inhibitor, or simultaneous administration of separate compositions such that one composition contains the CCR2 antagonist and another composition contains the PD-1 and/or PD-L1 inhibitor.

Co-administration includes administering the CCR2 antagonist of the present invention within 0.5, 1, 2, 4, 6, 8, 10, 12, 16, 20, or 24 hours of the PD-1 and/or PD-L1 inhibitor of the present invention. Co-administration also includes administering simultaneously, approximately simultaneously (e.g., within about 1, 5, 10, 15, 20, or 30 minutes of each other), or sequentially in any order. Moreover, the CCR2 antagonist and PD-1 and/or PD-Linhibitor can each be administered once a day, or two, three, or more times per day so as to provide the preferred dosage level per day.

### Kits

In some aspects, provided herein are kits containing a CCR2 chemokine receptor antagonist and a PD-1 and/or PD-L1 inhibitor disclosed herein that are useful for treating a cancer. A kit can contain a pharmaceutical composition containing a CCR2 chemokine receptor antagonist compound, e.g., a small molecule inhibitor of CCR2 and a pharmaceutical composition containing an PD-1 and/or PD-L1, e.g., an antibody inhibitor. In some instances, the kit includes written materials e.g., instructions for use of the compound, antibody or pharmaceutical compositions thereof. Without limitation, the kit may include buffers, diluents, filters, needles, syringes, and package inserts with instructions for performing any methods disclosed herein.

Suitable CCR2 chemokine receptor antagonist and PD-1 and/or PD-L1 inhibitors include the compounds described herein.

### EXAMPLES

### Example 1

Cell Culture

KR158 glioma cells were maintained in Dulbecco's modified Eagle's medium (DMEM) supplemented with 10% heat-inactivated fetal bovine serum (FBS) and 1% penicillin-streptomycin. 005 GSC glioma cells were cultured as neurospheres in serum free Advanced DMEM/F12 medium supplemented with 2 mM L-glutamine, 1% N2 supplement, 2 mg/mL heparin, 0.5% penicillin-streptomycin, 20 ng/mL recombinant human EGF, and 20 ng/mL recombinant human FGF-basic. GL261 glioma cells were cultured in Roswell Park Memorial Institute (RPMI)-1640 supplemented with 10% FBS, 4 mM L-glutamine, and 1% penicillin-streptomycin. All cells were grown in a humidified incubator at 37° C. with 5% CO₂. DMEM, Advanced DMEM/F12, N2 supplement, EGF, bFGF, L-glutamine and antibiotics were obtained from Gibco-BRL (Invitrogen, Carlsbad, Calif.). Heparin was purchased from Sigma-Aldrich (St Louis, Mo.). FBS was from HyClone (Thermo Scientific, Waltham, Mass.).

Animals:

Wild type (WT) C57BL/6, Ccr2 deficient (Cr2^{RFP/RFP}[B6.129(Cg)-Ccr2^{tm2.1Ifc}/J]), and Cx3cr1 deficient (Cx3cr1^{GFP/GFP}[B6.129P-Cx3cr1^{tm1Litt}/J]) mice were obtained from Jackson Laboratory (Bar Harbor, Me.). Ccr2^{RFP/WT}/Cx3cr1^{GFP/WT} mice (double knock-in) were generated via in house breeding.

Intracranial Injection of GBM Cells:

Animals were anesthetized using isoflurane, and administered analgesia prior to cell injection. While under anesthesia, the surgical site was prepared, a 2-3 mm incision was made at the midline of the skull, and a small burr hole was drilled 1 mm posterior and 2 mm lateral from bregma. KR158 glioma (7.5×10⁴), 005 GSC glioma cells (5×10⁴), or GL261 glioma cells (0.75-1×10⁵) in a total volume not exceeding 2 μl were injected 3 mm deep into the right cerebral hemisphere. The surgical site was closed via suture, and the animal was placed into a warm cage for post-surgical monitoring.

Drug Treatments:

Compound 3 was delivered for 21 days, beginning on day 7 after tumor cell injection, by oral gavage at a dose of 90 mg/kg, twice daily. Animals also received either anti-PD-1 (catalog # BE0146, clone RMPI-14, BioXcell) or non-immune IgG (catalog # BE0089, clone 2A, BioXcell) treatment injected intraperationally alone or in combination with Compound 3, every third day beginning 7 days after implantation for a total of 5 doses (loading dose of 500 ug/100 uL, followed by 4 doses of 200 ug/100 uL). A control group of mice was treated in parallel to drug administration with vehicle and/or non-immune IgG. The number of mice used in each treated group is indicated within the figure legends.

Kaplain-Meier Analysis for Survival:

For Kaplan-Meier survival analysis, percentages of surviving mice in the various groups were recorded daily after either KR158 or 005 GSC glioma cell implantation, until endpoint or 100-120 days, at which time all remaining animals were euthanized. Humane endpoint was defined by a lack of physical activity, body weight reduction >15%, loss of righting response, body score <2, onset of seizures, or signs of pain/distress. Log-rank test was used to determine significance between the experimental groups.

Bone Marrow Imaging:

Mice were euthanized, after which femurs were removed and fixed in 4% PFA at 4° C. for 3 days with constant agitation. Following fixation, femurs were decalcified using 14% ethylenediaminetetraacetic acid (EDTA)/9% ammonium hydroxide (w/v, pH 7.1) decalcifying solution at 4° C. for 3 days with constant agitation, changing solution every 24 hours. Bones were then washed in phosphate buffered saline (PBS) for 2 hours then soaked in 30% sucrose at 4° C. overnight with constant agitation. Bones were then embedded in optimal cutting temperature (OCT) medium, sectioned, and analyzed by fluorescent microscopy.

Immunohistochemistry:

For immunohistochemistry, brain sections from Ccr2^{RFP/WT} and Ccr2^{RFP/RFP} mice were first permeabilized with 0.5% Triton X-100 for 15 min at room temperature followed by heating slides (immersed in a boiling water bath for 25 min) in a buffer containing 10 mM sodium citrate, 0.05% Tween 20, pH 6.0. Slides were then cooled to room temperature for 20 min, washed with PBS three times, and blocked with 10% goat serum in PBS for 30 min. The sections were incubated in primary antibodies at 4° C. overnight. Antibodies used are listed in Supplement table 1. The following day, sections were washed three times with PBS and incubated subsequently in goat anti-rat Alexa 594 (dilution 1:1000, BD Pharmingen). The sections were then washed three times with PBS, counterstained with DAPI, and imaged by fluorescent microscopy.

Flow Cytometry:

Mice were euthanized using CO₂ asphyxiation at experimental endpoint. Following euthanasia, the spleen and femur were removed and placed in PBS. The animal was subsequently perfused with 0.9% saline via cardiac puncture and the brain removed. Bone marrow was extracted by flushing with PBS using a 25G needle. Splenocytes were liberated by fracturing the organ capsule between glass slides and rinsing with fluorescence-activated cell sorter washing buffer (PBS and 1% FBS, FACS), followed by needle puncture with an 18G needle. Splenocytes were then collected by centrifugation (4° C., 380G, 5 minutes), re-suspended in FACS and passed through a 50 μm cell strainer. Splenocytes and bone marrow samples were then centrifuged (4° C., 380G, 5 minutes), re-suspended in ACK lysis buffer (Gibco, Invitrogen, Carlsbad, Calif.), and incubated for 1.5 minutes at room temperature (Splenocytes) or 10 minutes (bone marrow) at 4° C. At end of incubation, lysis was halted using 9 mL FACS buffer. Cells were then centrifuged (4° C., 380G, 5 minutes), re-suspended in PBS, and collected in 1.5 mL microcentrifuge tubes. Tumors were then excised from brains and minced using a razor blade. Tissue was suspended in 4° C. Accumax dissociation solution (Innovative Cell Technologies, San Diego Calif.) and incubated at 37° C. for 5 minutes followed by 5 minutes of agitation at room temperature. Cells were then passed through a 70 μm strainer, centrifuged (4° C., 380G, 5 minutes), and re-suspended in 4 mL 70% Percoll (70% Percoll and 1% PBS in RPMI 1640 cell medium). This cell suspension was then gently layered beneath a 37% Percoll layer (4 mL, 37% Percoll and 1% PBS in RPMI 1640 cell medium) using an 18G needle, centrifuged (30 minutes, room temperature, 500G), the interface removed and placed into a 1.5 mL microcentrifuge tube. All cells were then washed with ice cold PBS, counted by trypan blue exclusion, aliquoted to 1×10⁶ cells/100 PL, and blocked using 0.5 μg anti-mouse CD16/32 (101320, Biolegend, San Diego Calif.) for 30 minutes at 4° C. Subsequently, cells were stained for markers of interest for 30 minutes at 4° C. Cells were then washed twice in ice cold PBS and either fixed in 4% PFA for 30 minutes and re-suspended in FACS buffer, or left unfixed if isolated from reporter mice. Stained samples were analyzed using single color compensation on either a BD LSR Fortessa flow cytometer (BD Biosciences, San Jose, Calif.) or a SONY SP6800 spectral analyzer (SONY, San Jose, Calif.), and quantified using FCS express software (De Novo software, Glendale, Calif.).

Statistical Analysis:

Student's t-test was performed in SigmaPlot (SigmaPlot, London, UK) as indicated in the results. p-values were calculated using Student's t-test with two-tailed distribution. Survival data were subjected to log-rank test using GraphPad Prism 5 software (GraphPad Software, La Jolla, Calif.) to determine statistically significant differences between groups. A p-value <0.05 was considered significant and is indicated by symbols depicted in the figures and figure legends.

Results

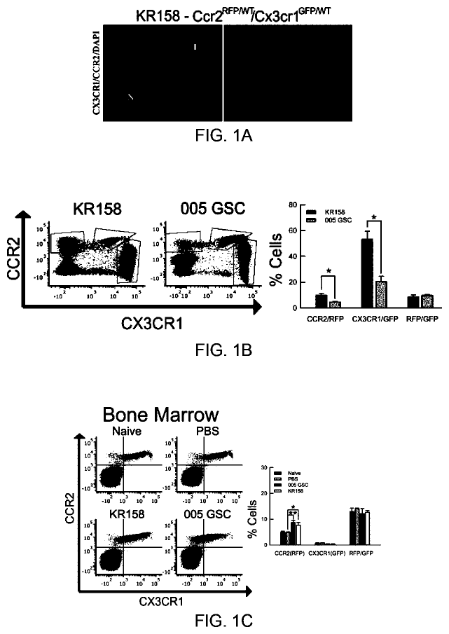

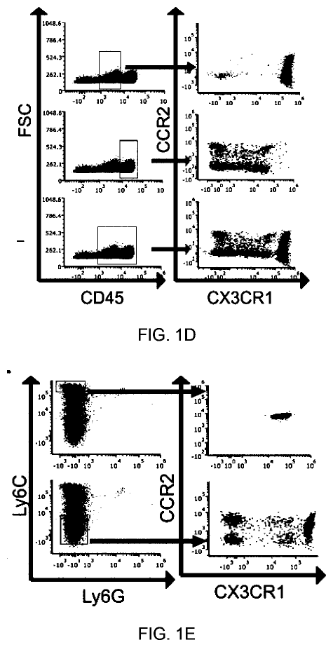

CCR2⁺ cells do not represent the sole myeloid cell type present in gliomas, as CX3CR1 CNS resident microglia are known to infiltrate as well. As a means to investigate the glioma presence of these chemokine receptor-expressing myeloid cell populations, we employed double transgenic mice which carry RFP in place of the CCR2 gene (CCR2^{RFP/WT}) and GFP in place of CX3CR1 (CX3CR1^{GFP/WT}) as knock-in alleles, enabling direct surveillance of CCR2⁺ and CX3CR1⁺ cells. Two therapy resistant murine glioma models were employed including the high-grade glioma KR158 model and the recently reported glioblastoma stem-like cell 005 GSC model. Fluorescent imaging confirmed the presence of both CCR2⁺ and CX3CR1⁺ cells within KR158 tumors (FIG. 1A). Flow cytometry analysis identified tumor-associated CCR2⁺ and CX3CR1⁺ cells in both glioma models. However, the presence of both populations was significantly higher in KR158 tumors (CCR2⁺, p=0.048; CX3CR1⁺, p=0.012) (FIG. 1B). Analysis of the bone marrow revealed a significant increase in CCR2⁺ cells upon either KR158 (p=0.032) or 005 GSC (p=0.001) tumor implantation, with no change in this cell population as a result of PBS injection (FIG. 1C). The GFP⁺/RFP⁺ cell population (CCR2⁺/CX3CR1⁺) was unchanged in the bone marrow of the tumor-bearing animals.

We next sought to characterize the myeloid marker phenotypes of the CCR2⁺ and CX3CR1⁺ populations in the tumor microenvironment. In order to investigate these populations, tumor infiltrates from glioma-bearing CCR2^{RFP/WT};CX3CR1^{GFP/WT} mice were subjected to flow cytometry analysis of CD45, CD11b, Ly6C, and Ly6G. Two distinct CD45⁺ populations were identified, designated CD45^{low} and CD45^{hi} (FIG. 1D). Analysis of these populations revealed CD45^{low} events (FIG. 1D upper) represent a cell population that is primarily CX3CR1⁺, likely representing microglia. CD45^{hi} (FIG. 1D middle) events represent a more heterogeneous cell population consisting of CCR2⁺, CX3CR1+, and CCR2⁻/CX3CR1⁻ cells. Murine monocytic MDSCs are typically classified as CD11b⁺/Ly6C^{hi}/Ly6G⁻. To examine the heterogeneous CD45^{hi} population, CCR2⁺ and CX3CR1 populations were scrutinized by expression of CD11b/Ly6C/Ly6G. Flow cytometric analysis of Ly6C/Ly6G noted three distinct Ly6C populations: negative, intermediate, and high (FIG. 1E). Ly6G expression was minimal in the tumors. Ly6C^{hi} events (FIG. 1E upper) represented a cell population that is primarily CCR2⁺/CX3CR1⁺, while Ly6C⁻ (FIG. 1E lower) events consist of CCR2⁺, CX3CR1⁺, and CCR2⁻/CX3CR1⁻ cells. Ly6C^{inter} events were determined to be CCR2/CX3CR1 double positive. Similar analysis within bone marrow isolates revealed four distinct populations: negative, Ly6C^{inter}/Ly6G⁻, Ly6C^{hi}/Ly6G⁻, and Ly6C^{inter}/Ly6G⁺. Ly6C^{hi}/Ly6G⁺ events were primarily CCR2⁺/CX3CR1⁺, while Ly6C⁻/Ly6G⁻, Ly6C^{inter}/Ly6G⁻, Ly6C^{inter}/Ly6G⁺ events were predominantly CCR2⁻/CX3CR1⁻. Additional flow cytometry analysis of CCR2- and CX3CR1-expressing cells determined that CCR2⁺/CX3CR1⁻ cells are MHCII⁺/F4/80⁻/CD11c⁺/CD11b^{lo}, CCR2⁺/CX3CR1⁺ cells are MHCII⁺/F4/80⁺/CD11c⁺/CD11b^{hi}, and CCR2⁻/CX3CR1⁺ cells are MHCII⁺/F4/80⁺/CD11c⁻/CD11b^{medium}. Taken together, invading myeloid cells expressing the two chemokine receptors within the tumor microenvironment are predominantly CCR2⁺ or CCR2⁺/CX3CR1⁺ double positive, while resident myeloid-like cells are predominantly CX3CR1⁺.

CCR2 Deficiency Unmasks an Anti-PD-1 Effect in Immune Checkpoint Inhibitor Resistant Glioma.

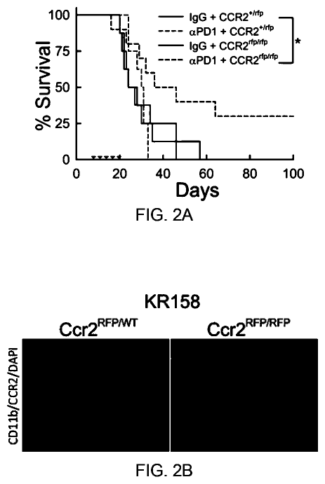

To establish a role of CCR2 in glioma and the potential impact of disrupting this receptor on the efficacy of immune checkpoint inhibitors, the effect of anti-PD-1 monotherapy in CCR2-sufficient and -deficient mice was evaluated. KR158 tumor bearing (n=8-10/group) CCR2^{RFP/WT} or CCR2^{RFP/RFP} mice were dosed with anti-PD-1 starting at day 7 as described in the methods and followed until humane end point (FIG. 2A). Survival analysis indicated no change in either median or durable survival due to CCR2 deficiency alone or anti-PD-1 monotherapy as compared to control groups. However, when anti-PD-1 was administered to CCR2-deficient mice, a significant increase (p=0.035) in overall durable survival was observed; differences in median survival between anti-PD-1 monotherapy treated strains (24 vs. 35 days) did not reach statistical significance. For proof of concept in high mutational burden tumors, it was found CCR2 deficiency also augmented PD-1 blockade in GL261 tumor bearing animals, with differential outcomes based on initial treatment time and total dosing of the antibody. Indeed, the variation in responses of GL261 gliomas to anti-PD-1 monotherapy is known.

CCR2 Deficiency has Reciprocal Effects on Presence of MDSCs in Tumor and Bone Marrow

Imaging analysis of CCR2 promoter driven RFP and staining for the myeloid marker CD11b confirmed the presence of CCR2⁺ myeloid derived cells within KR158 gliomas (FIG. 2B). The presence of these cells was reduced in KR158 tumors from CCR2-deficient mice. Fluorescence imaging of bone marrow revealed significantly elevated CCR2/RFP signal (reported as pixel density versus area of the cross section) in non-tumor bearing CCR2 deficient mice (p=0.029) as compared to heterozygous controls. Further elevation was observed in both CCR2^{RFP/WT} (p=0.011) and CCR2^{RFP/RFP} (p=0.036) following KR158 tumor implantation (FIG. 2C).

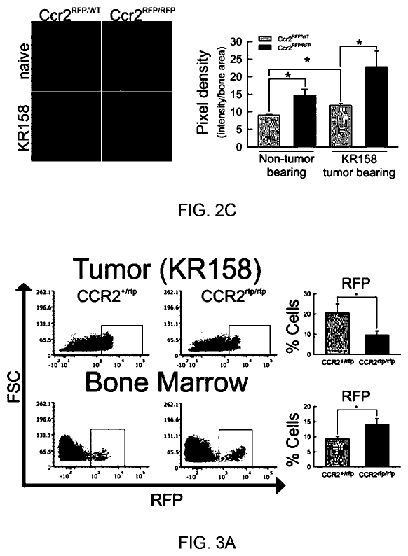

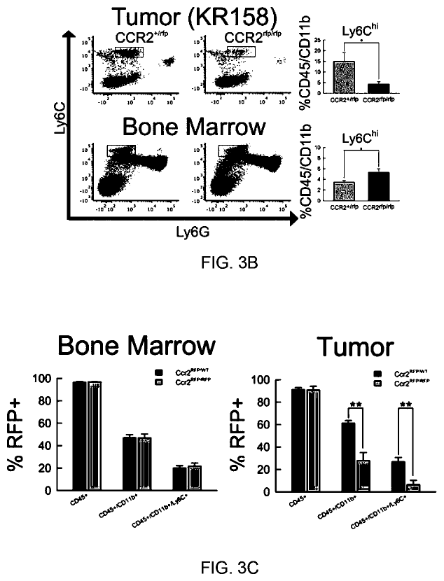

Flow cytometry analysis of the tumor-associated RFP⁺ cell population revealed a statistically significant decrease (p=0.047) of this population, while similar analysis of bone marrow showed a significant increase (p=0.024) (FIG. 3A) in CCR2 deficient tumor bearing mice. Not all CCR2⁺ cells were found to be Ly6C+. In order to more accurately examine the effect of CCR2 deficiency on the immune suppressive cell population of these mice, flow cytometry analysis of immune cells isolated from tumors and bone marrow of CCR2^{RFP/WT} and CCR2^{RFP/RFP} mice was performed. Analysis revealed a statistically significant reduction (p=0.039) of MDSCs (CD45^{hi}/CD11b⁺/Ly6C^{hi}) within KR158 tumors with a concomitant increase (p=0.020) in bone marrow (FIG. 3B). Additionally, investigation of this population in the periphery was performed and a significant reduction (p=0.048) in the MDSC population present within spleens of tumor bearing animals was evident. The proportion of RFP⁺ cells that are also Ly6C^{hi} within the bone marrow is unchanged by CCR2 deficiency (FIG. 3C). However, when this proportion was determined in tumors, a marked reduction (p=0.007) of this population was noted with CCR2 deficiency.

Despite a noted reduction in MDSCs within tumors, an increase in CD4⁺ T-cells (p=0.031) was observed while the population of CD8⁺ T-cells remained unaltered by CCR2 knockout. A significant increase (p=0.003) of the ratio of CD8⁺ T-cells/MDSCs was evident within tumors derived from CCR2 deficient mice.

CCR2 Antagonist Compound 3 Enhances an Anti-PD-1 Effect to Improve Survival.

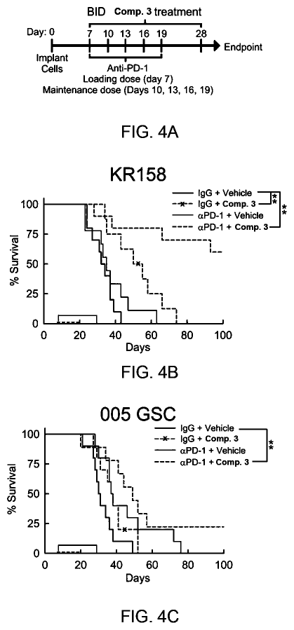

The effect of an orally active, high affinity CCR2 antagonist, Compound 3 against gliomas when combined with anti-PD-1 therapy was evaluated. To determine the effect on survival, KR158 glioma bearing mice were treated with anti-PD-1 and/or Compound 3 and followed to humane endpoint. Control and anti-PD-1 monotherapy-treated animals showed no difference in median or durable survival. In contrast, Compound 3 monotherapy increased (p=0.002) median survival time (32 days vs. 50 days), while combination treatment resulted in a significant durable survival advantage over control (p=0.001) and Compound 3 single treatment (p=0.001) (FIG. 4B). Median survival of 005 GSC tumor bearing animals was increased (30 vs. 49 days, p=0.005) with combination treatment, though no Compound 3 monotherapy effect was observed (FIG. 4C).

Compound 3 Impedes Invasion of MDSC into Tumors and Prevents Egress from Bone Marrow.

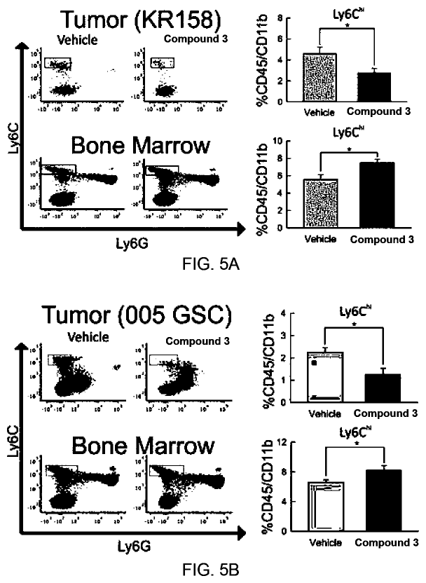

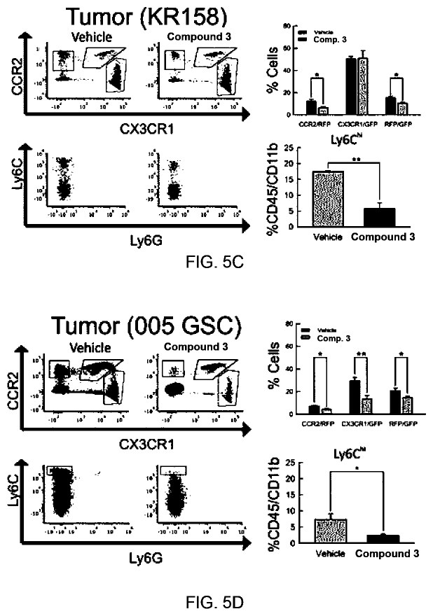

Similar to findings in CCR2 deficient mice, flow cytometry analysis of Compound 3 treated KR158 bearing animals revealed a decrease (p=0.038) in the population of CD45^{hi}/CD11b⁺/Ly6C^{hi} cells within the tumor microenvironment (FIG. 5A). A significant increase (p=0.028) of this population was observed in bone marrow. Analysis of 005 GSC tumor bearing animals mirrors the results observed with KR158 gliomas, i.e. a significant reduction (p=0.015) in the Ly6C^{hi} cell population within the tumors, and a concomitant increase (p=0.028) of this population in the bone marrow was seen (FIG. 5B).

The effect of Compound 3 treatment on the three CCR2 and CX3CR1 expressing subpopulations was evaluated. KR158 or 005 GSC bearing CCR2^{RFP/WT};CX3CR1^{GFP/WT} mice were treated with either vehicle or Compound 3. Immune cell populations were subsequently isolated and subjected to flow cytometry analysis of CCR2/RFP and CX3CR1/GFP expression, as well as for CD45, CD11b, Ly6C and Ly6G. Analysis of KR158 tumors revealed a significant decrease (p=0.003) in RFP⁺ i.e. CCR2⁺/CX3CR1⁻ cells with Compound 3 treatment. Similarly, CCR2⁺/CX3CR1⁺ reported a decrease (p=0.032) with Compound 3 treatment (FIG. 5C upper). Consistent with previous results, Compound 3 treatment reduced (p=0.004) CD45^{hi}/CD11b⁺/Ly6C^{hi} cells within KR158 tumors (FIG. 5C lower). Parallel analysis was performed in 005 GSC glioma-bearing animals. A significant reduction of CCR2 single positive (p=0.003), CX3CR1⁺ (p=0.003), as well as CCR2/CX3CR1 double positive (p=0.042) events (FIG. 5D upper) were observed in tumors from Compound 3-treated mice. Analysis of CD45^{hi}/CD11b⁺/Ly6C^{hi} cells within 005 GSC tumors also showed a reduction (p=0.020) in Ly6C^{hi} events with Compound 3 treatment (FIG. 5D lower).

Compound 3/Anti-PD-1 Combination Therapy Reduces Exhaustion in Intratumoral T-Cells.

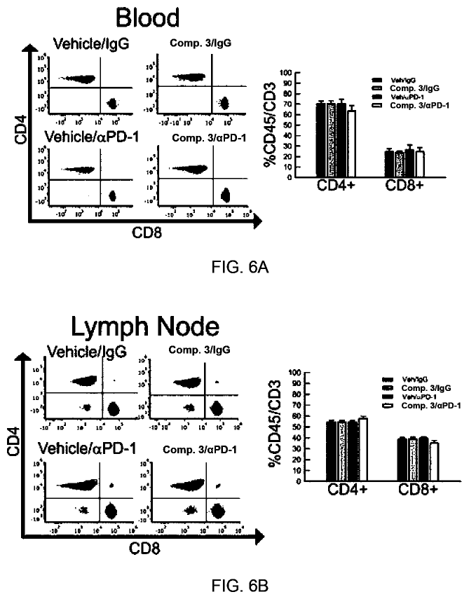

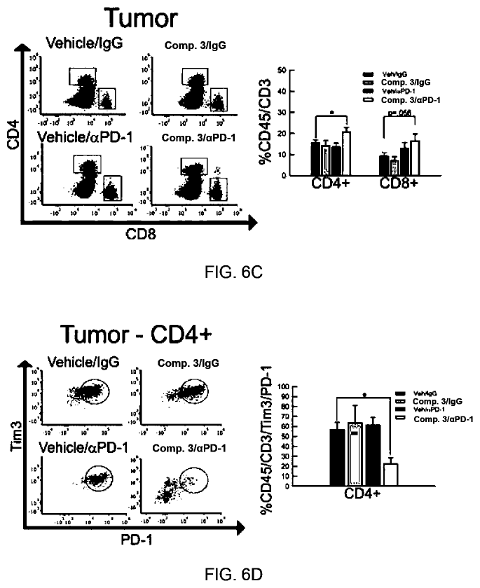

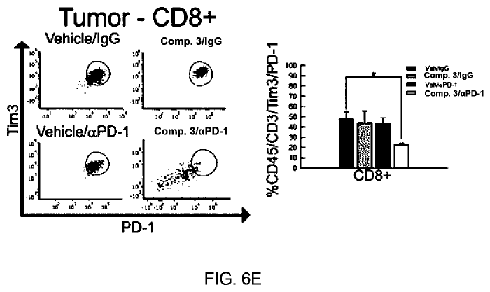

The effects of combination therapy on T cell populations in 005 GSC glioma-bearing wild type mice were evaluated. Peripheral CD4⁺ and CD8⁺ T-cell populations in blood (FIG. 6A) and lymph nodes (FIG. 6B) were not impacted by any of the treatments. A significant increase in tumor infiltrating CD45⁺/CD3⁺/CD4⁺ T-cells was noted with combination therapy (p=0.044) while a trend (p=0.056) toward increased percentage of CD45⁺/CD3⁺/CD8⁺ T cells was observed (FIG. 6C). Neither of the monotherapies produced changes in these tumor-infiltrating T-cell populations. Examination of T-cell exhaustion markers (PD-1⁺/Tim3⁺) on CD4⁺ and CD8⁺ T cells within tumors derived from all treatment groups determined that only the [Compound 3/anti-PD-1 combination therapy produced significant reductions of CD45⁺/CD3⁺/PD-1⁺/Tim3⁺/CD4⁺ (FIG. 6D, p=0.029) and CD45⁺/CD3⁺/PD-1⁺/Tim3⁺/CD8⁺ (FIG. 6E, p=0.011) T-cells. These data suggest combination therapy results in enhanced tumor infiltration of lymphocytes that are less dysfunctional.

Discussion

Since the inclusion of temozolomide into the standard of care regimen for GBM, little progress has been made in the development of effective treatments for this disease. Stagnating survival rates underscore the need for next generation approaches for the treatment of GBM. While immunotherapy based approaches have been attempted, most clinical trials involving these modalities have failed to report significant outcomes. MDSCs are known to potentiate immune-suppression in GBM and may contribute to the failure of immune therapies for gliomas. Blocking CCR2 by either gene deletion or pharmacological antagonism was able to unmask efficacy of immune checkpoint blockade in two clinically relevant murine glioma models. The data suggest that the enhanced survival is a consequence of reduced MDSCs within the glioma microenvironment, a concomitant increase of this cell population within bone marrow, and an increase in functional tumor infiltrating lymphocytes.

Disruption of CCR2 not only leads to reduced MDSCs within tumors, but an associated accumulation of these cells in the bone marrow. A role for CCR2 in mobilization of leukocytes from the bone marrow likely involves interactions with another chemokine receptor, CXCR4. The egress of CCR2⁺ cells from the bone marrow and influx into the tumors may be mediated by any known ligand for CCR2. In addition to CCL2, MCP-3 (CCL7) has been shown to be integral in migration of CCR2⁺ monocytes out of the bone marrow (47).

MDSCs have potential for wide-ranging impacts on T-cell activation and proliferation. The effects are exerted via an array of mechanisms including Arg-1/iNOS expression, ROS production, and recruitment of T-regulatory cells. Studies have suggested that infiltration of MDSCs into the GBM microenvironment is associated with a reduction in infiltrating lymphocytes. Additionally, it has been reported that PD-1 blockade increases tumor T-cell infiltration in models of melanoma and colon cancer via an IFN-γ dependent mechanism. In the models used herein, CCR2 antagonist monotherapy had no impact on intra-tumoral T-cell populations, while PD-1 blockade alone only marginally increased CD8⁺ T-cells, though not significantly. Elevated populations of both CD4⁺ and CD8⁺ T-cells within 005 GSC tumors were observed with combination treatment. The increased T-cell populations may be due to increased infiltration or reduced T-cell death within tumors. Exhaustion has been shown to promote T-cell apoptosis via PD-1/PD-L1 axis, and therefore may contribute to loss of T-cells at the tumor site. Using PD-1/Tim3 double expression on CD4⁺ or CD8⁺ T-cells as a marker for exhaustion, it was determined that only the combination therapy was able to reduce the population of exhausted T-cells within the tumor. Given that anti-PD-1 treatment alone did not enhance survival in either model, and was able to only marginally increase intra-tumoral T-cell population, these data may suggest the reduced exhaustion with combination therapy may be driving improvement in overall survival.

To summarize, our data show that CCR2 deficiency augments anti-PD-1 treatment and unmasks a survival advantage in glioma bearing mice. These results are recapitulated with CCR2 antagonism in mice bearing either KR158 or 005 GSC murine glioma models. The use of anti-PD-1 resistant syngeneic murine models enhances the translational value of this study as compared to others that have relied on immune-deficient mice or anti-PD-1 responsive glioma models.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a central nervous system cancer in a subject, comprising:
administering to a subject in need thereof, a therapeutically effective amount of an immune checkpoint inhibitor and a compound of formula I: 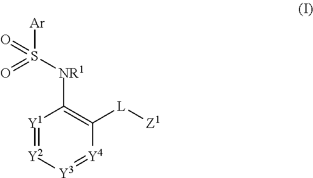
or a pharmaceutically acceptable salt thereof,
wherein
Ar is selected from the group consisting of substituted or unsubstituted C₆₋₁₀ aryl and substituted or unsubstituted 5- to 10-membered heteroaryl.
R¹ is selected from the group consisting of hydrogen, substituted or unsubstituted C₁₋₈ alkyl, substituted or unsubstituted C₂₋₆ alkenyl, substituted or unsubstituted C₂₋₆ alkynyl, and substituted or unsubstituted 3- to 10-membered heterocyclyl;
Y¹ is selected from the group consisting of —CR^{2a}—, —N—, and —N⁺(O)⁻—;
Y² is selected from the group consisting of —CR^{2b}—, —N—, and —N⁺(O)⁻—;
Y³ is selected from the group consisting of —CR^{2c}—, —N—, and —N⁺(O)⁻—;
R^{2a}, R^{2b}, and R^{2c} are each independently selected from the group consisting of hydrogen, halogen, —CN, —C(O)R³, —O₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³NR³R⁴, —NR³C(O)R⁴, —NR³C(O)OR⁴, —NR³S(O)₂R⁴, —NR³C(O)NR⁴R⁵, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, and substituted or unsubstituted 5- to 10-membered heteroaryl;
R³, R⁴, and R⁵ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R³ and R⁴, R⁴ and R⁵ or R³ and R⁵ may, together with the atoms to which they are attached, form a substituted or unsubstituted 5-, 6-, or 7-membered ring;
Y⁴ is selected from the group consisting of —N— and —N⁺(O)⁻—;
L is selected from the group consisting of a bond, —O—, —S—, —S(O)—, —S(O)₂—, —CR⁶R⁷, —NR⁸—, —C(O)—, —C(O)NR⁸—, and —NR⁸C(O)—;
R⁶ and R⁷ are each independently selected from the group consisting of hydrogen, halogen, substituted or unsubstituted C₁₋₈ alkyl, substituted or unsubstituted 3- to 10-membered heterocyclyl, substituted or unsubstituted C₂₋₆ alkenyl, substituted or unsubstituted C₂₋₆ alkynyl, —CN, —OR⁹, —NR¹⁰R¹¹, —S(O)R⁹, and —S(O)₂R⁹;
R⁶ and R⁷ may, together with the carbon atom to which they are attached, form substituted or unsubstituted C₃₋₈ cycloalkyl or substituted or unsubstituted 3- to 10-membered heterocyclic ring;
R⁹ is selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R¹⁰ and R¹¹ are each independently selected from the group consisting of substituted or unsubstituted C₁₋₈ alkyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted C₂₋₈ alkenyl, and substituted or unsubstituted C₂₋₈ alkynyl;
R¹⁰ and R¹¹ of NR¹⁰R¹¹ may, together with the nitrogen, form substituted or unsubstituted 3- to 10-membered heterocyclyl;
R⁸ is selected from the group consisting of hydrogen, C(O)R¹², S(O)₂R¹², CO₂R¹², substituted or unsubstituted C₁₋₈ alkyl, substituted or unsubstituted 3- to 10-membered heterocyclyl, substituted or unsubstituted C₂₋₆ alkenyl, and substituted or unsubstituted C₂₋₆ alkynyl;
R¹² is selected from the group consisting of substituted or unsubstituted C₁₋₈ alkyl, substituted or unsubstituted C₂₋₆ alkenyl, substituted or unsubstituted C₂₋₆ alkynyl, substituted or unsubstituted 3- to 10-membered heterocyclyl, substituted or unsubstituted C₆₋₁₀ aryl, and substituted or unsubstituted 5- to 10-membered heteroaryl;
Z¹ is selected from the group consisting of substituted or unsubstituted C₆₋₁₀ aryl, substituted or unsubstituted 5- to 10-membered heteroaryl, substituted or unsubstituted 3- to 10-membered heterocyclyl, and —NR¹³R¹⁴;
R¹³ and R¹⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted (C₁₋₄ alkyl)-(C₆₋₁₀ aryl), and substituted or unsubstituted (C₁₋₄ alkyl)-(5- to 10-membered heteroaryl);
R¹³ and R¹⁴ may, together with the nitrogen, form a substituted or unsubstituted 4-, 5-, 6-, or 7-membered heterocyclyl.

2. The method of claim 1, wherein the compound is of formula (Ic), or a pharmaceutically acceptable salt thereof, 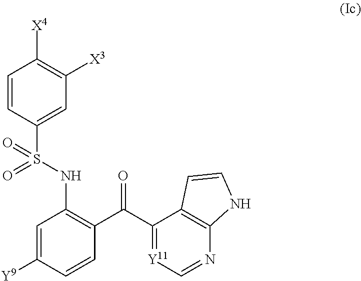
wherein Y¹¹ is —CH—, —N—, and —N⁺(O)⁻—.

3. The method of claim 1, wherein the compound is selected from the group consisting of: 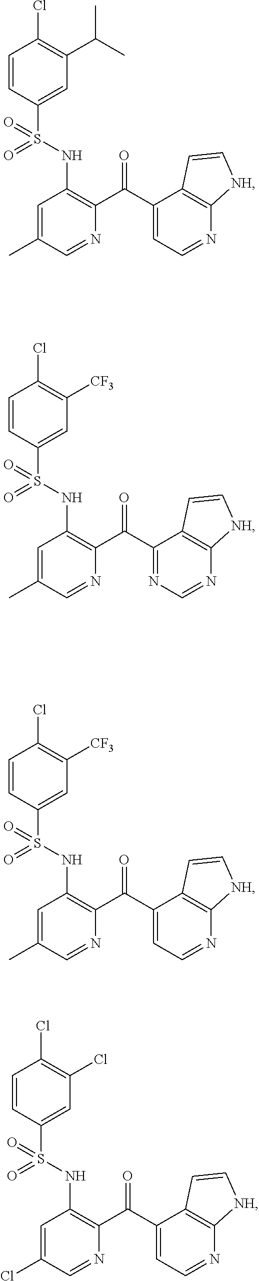 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1 ; O=C(c1ncc(Cl)cc1NS(=O)(=O)c1ccc(Cl)c(Cl)c1)c1ccnc2[nH]ccc12] 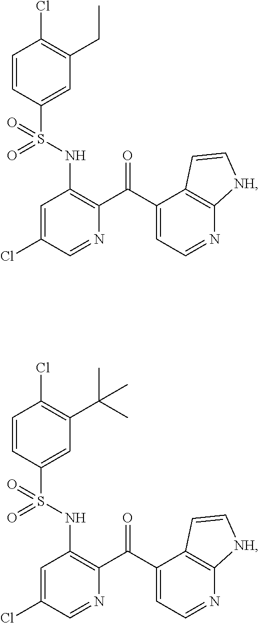 [SMILES: CCc1cc(S(=O)(=O)Nc2cc(Cl)cnc2C(=O)c2ccnc3[nH]ccc23)ccc1Cl ; CC(C)(C)c1cc(S(=O)(=O)Nc2cc(Cl)cnc2C(=O)c2ccnc3[nH]ccc23)ccc1Cl]
and pharmaceutically acceptable salts thereof.

4. The method of claim 1, wherein the immune checkpoint inhibitor is a monoclonal antibody.

5. The method of claim 1, wherein the immune checkpoint inhibitor is an anti-PD-1 antibody or an anti-PD-L1 antibody.

6. The method of claim 1, wherein the central nervous system cancer is glioma.

7. The method of claim 1, wherein the central nervous system cancer is glioblastoma.

8. The method of claim 1, wherein the central nervous system cancer is characterized as being CCR2⁺.

9. The method of claim 1, wherein the administering of the compound of formula I, or a pharmaceutically acceptable salt thereof, promotes a decrease in CD45^{hi}/CD11b⁺/Ly6C^{hi} cells in a tumor microenvironment and promotes an increase in CD45^{hi}/CD11b⁺/Ly6C^{hi} cells in bone marrow.

10. The method of claim 1, wherein the administering to the patient of the immune checkpoint inhibitor and the compound of formula I or a pharmaceutically acceptable salt thereof promotes an infiltration of a population of T-cells into a tumor microenvironment in the subject.

11. The method of claim 10, wherein the population of T-cells comprises a subpopulation of T-cells characterized as being CD45⁺/CD3⁺/CD4⁺.

12. The method of claim 10, wherein the population of T-cells comprises a subpopulation of T-cells characterized as being CD45⁺/CD3⁺/CD8⁺.

13. The method of claim 10, wherein the compound of Formula I or a pharmaceutically acceptable salt thereof is provided as a pharmaceutical composition for oral administration.

14. The method of claim 1, wherein the effective amount of the compound of Formula I or a pharmaceutically acceptable salt thereof is from 50 mg to 300 mg.

15. A method of treating glioblastoma in a subject, comprising: administering to the subject in need thereof an effective amount of an immune checkpoint inhibitor and a compound selected from the group consisting of: 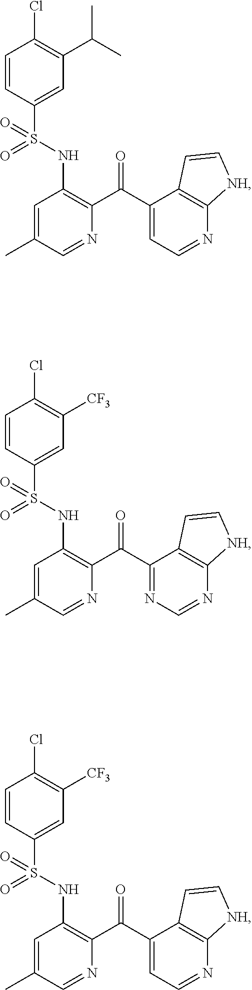 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1]
and pharmaceutically acceptable salts thereof.

16. The method of claim 15, wherein the immune checkpoint inhibitor is selected from the group consisting of pembrolizumab, nivolumab, IBI-308, mDX-400, BGB-108, MEDI-0680, SHR-1210, PF-06801591, PDR-001, GB-226, STI-1110, biosimilars thereof, biobetters thereof, and bioequivalents thereof.

17. The method of claim 15, wherein the immune checkpoint inhibitor is an anti-PD-1 antibody is selected from the group consisting of Nivolumab, Pembrolizumab, and Pidilizumab.

18. The method of claim 15, wherein the immune checkpoint inhibitor is a PD-L1 inhibitor is selected from the group consisting of durvalumab, atezolizumab, avelumab, BMS-936559, ALN-PDL, TSR-042, KD-033, CA-170, CA-327, STI-1014, KY-1003, biosimilars thereof, biobetters thereof, and bioequivalents thereof.

19. The method of claim 1, wherein the compound of Formula I, or a pharmaceutically acceptable salt thereof, and the immune checkpoint inhibitor are administered concomitantly.

20. The method of claim 1, wherein the compound of Formula I, or a pharmaceutically acceptable salt thereof, and the PD-1 inhibitor and/or the PD-L1 inhibitor are administered in a combination formulation.

21. The method of claim 1, wherein the compound of Formula I, or a pharmaceutically acceptable salt thereof, and the PD-1 inhibitor and/or the PD-L1 inhibitor are administered sequentially.

22. The method of claim 1, wherein the compound of Formula I, or a pharmaceutically acceptable salt thereof, is administered prior to administration of the PD-1 inhibitor and/or the PD-L1 inhibitor.

23. The method of claim 1, wherein the compound of Formula I, or a pharmaceutically acceptable salt thereof, is administered after the administration of the PD-1 inhibitor and/or the PD-L1 inhibitor.

24. The method of claim 1, wherein the compound of Formula I, or a pharmaceutically acceptable salt thereof, is administered orally and the PD-1 inhibitor and/or the PD-L1 inhibitor is administered intravenously.

25. The method of claim 1, wherein the subject is a human subject.

26. A pharmaceutical combination for treating glioblastoma in a patient, comprising:
a PD-1 and/or PD-L1 inhibitor; and
a compound of formula I: 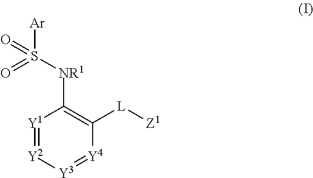
or a pharmaceutically acceptable salt thereof, wherein
Ar is selected from the group consisting of substituted or unsubstituted C₆₋₁₀ aryl and substituted or unsubstituted 5- to 10-membered heteroaryl.
R¹ is selected from the group consisting of hydrogen, substituted or unsubstituted C₁₋₈ alkyl, substituted or unsubstituted C₂₋₆ alkenyl, substituted or unsubstituted C₂₋₆ alkynyl, and substituted or unsubstituted 3- to 10-membered heterocyclyl;
Y¹ is selected from the group consisting of —CR^{2a}—, —N—, and —N⁺(O)⁻—;
Y² is selected from the group consisting of —CR^{2b}—, —N—, and —N⁺(O)⁻—;
Y³ is selected from the group consisting of —CR^{2c}—, —N—, and —N⁺(O)⁻—;
R^{2a}, R^{2b}, and R^{2c} are each independently selected from the group consisting of hydrogen, halogen, —CN, —C(O)R³, —CO₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³NR³R⁴, —NR³C(O)R⁴, —NR³C(O)OR⁴, —NR³S(O)₂R⁴, —NR³C(O)NR⁴R⁵, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, and substituted or unsubstituted 5- to 10-membered heteroaryl;
R³, R⁴, and R⁵ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R³ and R⁴, R⁴ and R⁵ or R³ and R⁵ may, together with the atoms to which they are attached, form a substituted or unsubstituted 5-, 6-, or 7-membered ring;
Y⁴ is selected from the group consisting of —N— and —N⁺(O)⁻—;
L is selected from the group consisting of a bond, —O—, —S—, —S(O)—, —S(O)₂—, —CR⁶R⁷—, —NR⁸, —C(O)—, —C(O)NR⁸—, and —NR⁸C(O)—;
R⁶ and R⁷ are each independently selected from the group consisting of hydrogen, halogen, substituted or unsubstituted C₁₋₈ alkyl, substituted or unsubstituted 3- to 10-membered heterocyclyl, substituted or unsubstituted C₂₋₆ alkenyl, substituted or unsubstituted C₂₋₆ alkynyl, —CN, —OR⁹, —NR¹⁰R¹¹, —S(O)R⁹, and —S(O)₂R⁹;
R⁶ and R⁷ may, together with the carbon atom to which they are attached, form substituted or unsubstituted C₃₋₈ cycloalkyl or substituted or unsubstituted 3- to 10-membered heterocyclic ring;
R⁹ is selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl;
R¹⁰ and R¹¹ are each independently selected from the group consisting of substituted or unsubstituted C₁₋₈ alkyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted C₂₋₈ alkenyl, and substituted or unsubstituted C₂₋₈ alkynyl;
R¹⁰ and R¹¹ of —NR¹⁰R¹¹ may, together with the nitrogen, form substituted or unsubstituted 3- to 10-membered heterocyclyl;
R⁸ is selected from the group consisting of hydrogen, C(O)R¹², S(O)₂R¹², CO₂R¹², substituted or unsubstituted C₁₋₈ alkyl, substituted or unsubstituted 3- to 10-membered heterocyclyl, substituted or unsubstituted C₂₋₆ alkenyl, and substituted or unsubstituted C₂₋₆ alkynyl;
R¹² is selected from the group consisting of substituted or unsubstituted C₁₋₈ alkyl, substituted or unsubstituted C₂₋₆ alkenyl, substituted or unsubstituted C₂₋₆ alkynyl, substituted or unsubstituted 3- to 10-membered heterocyclyl, substituted or unsubstituted C₆₋₁₀ aryl, and substituted or unsubstituted 5- to 10-membered heteroaryl;
Z¹ is selected from the group consisting of substituted or unsubstituted C₆₋₁₀ aryl, substituted or unsubstituted 5- to 10-membered heteroaryl, substituted or unsubstituted 3- to 10-membered heterocyclyl, and —NR¹³R¹⁴;
R¹³ and R¹⁴ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, substituted or unsubstituted 5- to 10-membered heteroaryl, substituted or unsubstituted (C₁₋₄ alkyl)-(C₆₋₁₀ aryl), and substituted or unsubstituted (C₁₋₄ alkyl)-(5- to 10-membered heteroaryl);
R¹³ and R¹⁴ may, together with the nitrogen, form a substituted or unsubstituted 4-, 5-, 6-, or 7-membered heterocyclyl.

27. The pharmaceutical combination of claim 26, wherein the compound is selected from: 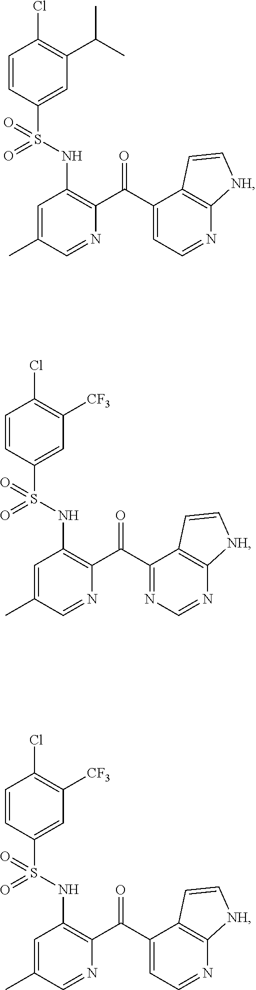 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1] 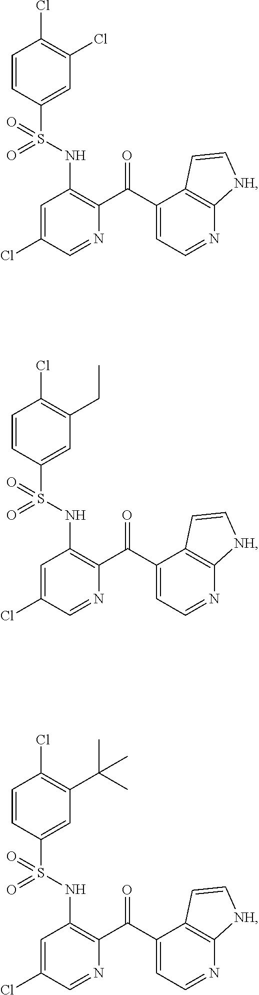 [SMILES: O=C(c1ncc(Cl)cc1NS(=O)(=O)c1ccc(Cl)c(Cl)c1)c1ccnc2[nH]ccc12 ; CCc1cc(S(=O)(=O)Nc2cc(Cl)cnc2C(=O)c2ccnc3[nH]ccc23)ccc1Cl ; CC(C)(C)c1cc(S(=O)(=O)Nc2cc(Cl)cnc2C(=O)c2ccnc3[nH]ccc23)ccc1Cl]
and pharmaceutically acceptable salts thereof.

28. The pharmaceutical combination of claim 26, wherein the compound of formula I is selected from the group consisting of: 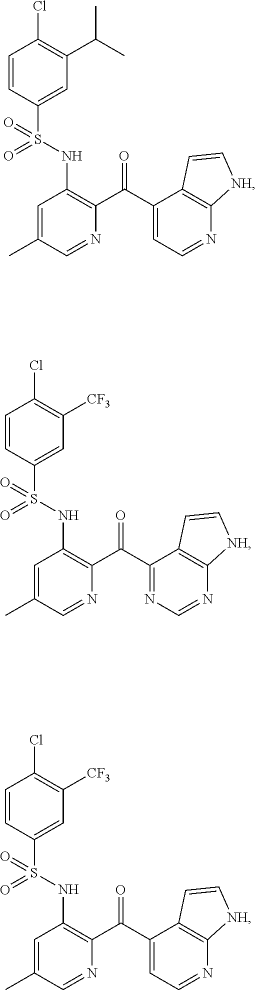 [SMILES: Cc1cnc(C(=O)c2ccnc3[nH]ccc23)c(NS(=O)(=O)c2ccc(Cl)c(C(C)C)c2)c1 ; Cc1cnc(C(=O)c2ncnc3[nH]ccc23)c(NS(=O)(=O)c2ccc(Cl)c(C(F)(F)F)c2)c1 ; Cc1cnc(C(=O)c2ccnc3[nH]ccc23)c(NS(=O)(=O)c2ccc(Cl)c(C(F)(F)F)c2)c1]
and pharmaceutically acceptable salts thereof.

29. The pharmaceutical combination of claim 26, wherein the combination comprises a fixed dose combination or separate doses.

30. The pharmaceutical combination of claim 26, wherein the pharmaceutical composition is formulated for intravenous administration.
